(19) United States
(12) Patent Application Publication
(10) Pub. No.: US 2022/0296868 A1
(43) Pub. Date: Sep. 22, 2022

(54) INTRACRANIAL DELIVERY OF MEDICINAL SOLUTION

(71) Applicant: InCube Labs, LLC, San Jose, CA (US)

(72) Inventors: Mir Imran, Los Altos Hills, CA (US); Mir Hashim, Fremont, CA (US)

(21) Appl. No.: 17/631,251

(22) PCT Filed: Jul. 30, 2020
(86) PCT No.: PCT/US20/44343

§ 371 Date: Jan. 28, 2022

Related U.S. Application Data
(60) Provisional application No. 62/881,875, filed on Aug. 1, 2019.

(51) Int. Cl. A61M 39/02

(52) U.S. Cl. CPC A61M 39/0247; A61M 2039/025; A61M 2039/0273; A61M 2039/0205; A61M 2039/027; A61M 2039/0267; A61M 2205/3334; A61M 2205/3344; A61M 25/0102

(57) ABSTRACT

Embodiments provide for intracranial delivery of a medicinal solution to the brain. A system includes a bun hole stopple for insertion in a cranial bun hole, a catheter, a connecting member, a connector tube, and a pump. The catheter is advanced through an opening in the stopple to a tissue site in the brain. A proximal portion of the catheter is fixed to an outer groove in the stopple to minimize movement of the catheter in the brain. The catheter, connecting member, connector tube, and pump are fluidically coupled together to create a flow path between the pump and a distal end of the catheter for infusion of the medicinal solution to the tissue site in the brain.

### CROSS-REFERENCE TO RELATED APPLICATIONS

This application claims priority to, and the benefit of, U.S. Provisional Patent Application No. 62/881,875 filed on Aug. 1, 2019 and titled Devices, Systems and Methods for Intracranial Delivery of Therapeutic Agents to Treat Brain Tumors, which is incorporated herein by reference in its entirety for all purposes.

### FIELD OF THE INVENTION

Embodiments relate to apparatuses, systems and methods for the treatment of adverse neurological conditions, such as for treatment of brain tumors or other cranial growths.

### BACKGROUND

Cancer of the brain and other nervous system cancers are a leading cause of death for men and women. For example, glioblastoma multiforme is the most commonly diagnosed primary brain tumor in adults, with an incidence of 2-3 cases per 100,000 population per year. This is a particularly lethal and fast-acting form of cancer, as the median life expectancy without treatment is approximately 4.5 months and the maximum life expectancy without treatment is generally considered to be 15 months.

Conventional therapies employed to treat undesired growths in the brain include surgical resection, radiotherapy, and chemotherapy. Therapies may be repeated, or two or more therapies may be used in combination or sequence. These conventional therapies each suffer well-known drawbacks and are often only marginally effective for treating aggressive growths such as glioblastoma.

Major drawbacks of surgical resection include risks generally associated with cranial and brain surgery, as well as the risks related to growth inaccessibility. Attempts to remove growths or portions of growths surrounded or intercalated in healthy brain tissue can cause injury to the healthy tissue, thus the physician and subject must choose either possible brain injury to fully resect the growth, or designating the growth or portions of the growth as inaccessible for resection. Inaccessibility is a particular problem for growths such as glioblastomas that invade many different areas of healthy tissue within the brain. Due to inaccessibility or other considerations, surgical removal of an entirety of a growth is not always practicable, and any non-removed portions of the growth can continue to grow.

With respect to radiotherapy and chemotherapy, a dosage required to kill growth cells generally also results in the killing of healthy cells. An additional complication with chemotherapy is that the blood-brain barrier often creates impediments to delivery into the brain by conventional delivery methods (e.g., intravenous (IV) infusion, subcutaneous injection, or oral delivery), thus requiring even higher doses of chemotherapy. Side effects from radiotherapy and chemotherapy can therefore be prohibitively severe, such as damage to vital areas of the brain resulting in a reduction of speech, motor skills, and cognitive skills. Further, many drugs are unable to cross the blood brain barrier in any meaningful amount. Additionally, for inoperable glioblastomas (which are many), the median survival time using a combination of radiotherapy and chemotherapy treatments is still only about 15 months.

### SUMMARY OF THE INVENTION

In an embodiment, a system for delivery of a medicinal solution to a tissue site in a brain of a subject includes a catheter, a cranial burr hole stopple, a connecting member, an anchoring element, and a connector tube. The catheter has a proximal end and a distal end, and defines at least one catheter lumen. The stopple defines a stopple opening structured for advancement of the catheter therethrough. The stopple includes a plug structured to be inserted into a burr hole in a cranium of the subject, the plug including at least one seal positioned on a wall of the stopple opening to form a fluidic seal with an exterior of the catheter. The stopple further includes a flange structured to engage an outer surface of a skull of the subject when the plug is inserted into the burr hole, the flange defining a flange opening on a side portion of the flange and defining at least one groove on a top portion of the flange, the at least one groove structured to engage and retain the catheter. The connecting member has a proximal end and a distal end, the distal end structured to be coupled to the proximal end of the catheter, the connecting member defining at least one connecting member lumen. The anchoring element engages the flange opening and is structured to secure the connecting member to the flange. The connector tube has a proximal end and a distal end, the distal end structured to be coupled to the connecting member proximal end, the connector tube defining at least one connector tube lumen. The at least one catheter lumen, the at least one connecting member lumen, and the at least one connector tube lumen are structured to provide at least one flow path, when assembled together, for delivery of the medicinal solution to the tissue site in the brain.

In an embodiment, a method for the intracranial delivery of an active agent to a brain to focally treat a growth in a cranium of a subject includes: positioning a burr hole stopple in a burr hole in the cranium, the burr hole stopple defining a sealable opening; advancing a catheter through the burr hole stopple opening such that a tip of the catheter is positioned at a tissue site in the brain within or in proximity to the growth, the catheter defining a fluidic lumen having at least one aperture at a distal portion of the catheter; affixing a proximal portion of the catheter to a fixation feature in the burr hole stopple; creating a flow path between the tissue site and a pump operatively coupled to a reservoir containing a medicinal solution comprising the active agent; and pumping the medicinal solution from the reservoir to the tissue site via the flow path; wherein a flow rate and a duration of delivery are selected based on an established model so as to achieve a selected steady state diffusion volume of medicinal solution in the brain tissue.

In an embodiment, a method for focally treating a brain growth includes: intracranially delivering to a tissue site in the brain, within or in proximity to the growth, a medicinal solution comprising an active agent which is degraded at a pH at or above that found in healthy brain tissue, wherein the solution comprises substantially no buffering agent; and wherein the active agent has a cytotoxic effect on cancerous tissue in the growth and is deactivated upon contact with or after entering into healthy brain tissue surrounding the growth.

Further details of these and other embodiments and aspects of the invention are described more fully below, with reference to the attached drawing figures.

### BRIEF DESCRIPTION OF THE DRAWINGS

FIG. 1A illustrates an embodiment of an intracranial drug delivery system.

FIG. 1B illustrates a diffusion volume of an active agent created by delivery of a medicinal solution to a delivery site in the brain using the embodiment of the system of FIG. 1A.

FIG. 1C is an enlarged view of FIG. 1A.

FIG. 1D is an enlarged view of FIG. 1B.

FIG. 2A illustrates an embodiment of a burr hole stopple.

FIG. 2B illustrates an embodiment of a burr hole stopple.

FIG. 3A, FIG. 3B, FIG. 3C, and FIG. 3D illustrate an embodiment of placement of a catheter using a stylette at a tissue site in the brain. FIG. 3A shows advancement of the stylette; FIG. 3B shows advancement of the catheter over the stylette; FIG. 3C shows placement of the catheter tip in the brain growth; and FIG. 3D shows the positioned catheter connected to the intracranial drug delivery system.

FIG. 4A illustrates an embodiment of an intracranial drug delivery system.

FIG. 4B illustrates an embodiment of an intracranial drug delivery system.

FIG. 5A illustrates an embodiment of a burr hole stopple including an inductive coil and associated circuitry operatively coupled to the coil.

FIG. 5B illustrates an embodiment of a burr hole stopple including an inductive coil and associated circuitry operatively coupled to the coil.

FIG. 5C illustrates an embodiment of communication between a burr hole stopple and an external communication device.

FIG. 6A illustrates an embodiment of communication between a burr hole stopple and a head covering.

FIG. 6B is a view illustrating an embodiment of the head covering of FIG. 6A.

FIG. 7 is a graph of medicinal solution flow rate versus time illustrating embodiments of different delivery regimens.

FIG. 8 is a graph illustrating the generation of different steady state diffusion volumes of an active agent for varying flow rates of medicinal solution infused into a tissue site in the brain.

FIG. 9 is a flow chart of an algorithm for focally treating a brain growth in a subject using modelled correlations of steady state diffusion volumes of an active agent versus flow rate of a medicinal solution.

### DETAILED DESCRIPTION

Described herein are techniques and devices used for intercranial drug delivery to treat brain growths and other neurological conditions. The intracranial drug delivery may be into brain tissue or into cerebrospinal fluid (CSF) in the cranium such as in ventricles of the brain.

Before discussing details of the techniques and devices for intercranial drug delivery, a few conventions are provided for convenience of the reader.

Various abbreviations are used herein for standard units, such as deciliter (dl), milliliter (ml), microliter (il), international unit (IU), centimeter (cm), millimeter (mm), kilogram (kg), gram (gm), milligram (mg), microgram (μg), millimole (mM), degrees Celsius (° C.), degrees Fahrenheit (° F.), millitorr (mTorr), hour (hr), or minute (min).

When used in the present disclosure, the terms “e.g.,” “such as”, “for example”, “for an example”, “for another example”, “examples of”, “by way of example”, and “etc.” indicate that a list of one or more non-limiting example(s) precedes or follows; it is to be understood that other examples not listed are also within the scope of the present disclosure.

As used herein, the singular terms “a,” “an,” and “the” may include plural referents unless the context clearly dictates otherwise. Reference to an object in the singular is not intended to mean “one and only one” unless explicitly so stated, but rather “one or more.”

The term “in an embodiment” or a variation thereof (e.g., “in another embodiment” or “in one embodiment”) refers herein to use in one or more embodiments, and in no case limits the scope of the present disclosure to only the embodiment as illustrated and/or described. Accordingly, a component illustrated and/or described herein with respect to an embodiment can be used in another embodiment (e.g., in another embodiment illustrated and described herein, or in another embodiment within the scope of the present disclosure and not illustrated and/or not described herein).

The term “component” refers herein to one item of a set of one or more items that together make up a device, formulation or system under discussion. A component may be in a solid, powder, gel, plasma, fluid, gas, or other form. For example, a device may include multiple solid components which are assembled together to structure the device and may further include a liquid component that is disposed in the device. For another example, a formulation may include two or more powdered and/or fluid components which are mixed together to make the formulation.

The term “design” or a grammatical variation thereof (e.g., “designing” or “designed”) refers herein to characteristics intentionally incorporated into a design based on, for example, estimates of tolerances related to the design (e.g., component tolerances and/or manufacturing tolerances) and estimates of environmental conditions expected to be encountered by the design (e.g., temperature, humidity, external or internal ambient pressure, external or internal mechanical pressure, external or internal mechanical pressure stress, age of product, physiology, body chemistry, biological composition of fluids or tissue, chemical composition of fluids or tissue, pH, species, diet, health, gender, age, ancestry, disease, tissue damage, shelf life, or the combination of such); it is to be understood that actual tolerances and environmental conditions before and/or after delivery can affect such designed characteristics so that different components, devices, formulations, or systems with a same design can have different actual values with respect to those designed characteristics. Design encompasses also variations or modifications to the design, and design modifications implemented after manufacture.

The term “manufacture” or a grammatical variation thereof (e.g., “manufacturing” or “manufactured”) as related to a component, device, formulation, or system refers herein to making or assembling the component, device, formulation, or system. Manufacture may be wholly or in part by hand and/or wholly or in part in an automated fashion.

The term “structured” or a grammatical variation thereof (e.g., “structure” or “structuring”) refers herein to a component, device, formulation, or system that is manufactured according to a concept or design or variations thereof or modifications thereto (whether such variations or modifications occur before, during, or after manufacture) whether or not such concept or design is captured in a writing.

The term “body” refers herein to an animalia body having a GI tract.

The term “subject” refers herein to a body into which an embodiment of the present disclosure is, or is intended to be, delivered. For example, with respect to humans, a subject may be a patient under treatment of a health care professional.

The term “fluid” refers herein to a liquid or gas, and encompasses moisture and humidity. The term “fluidic environment” refers herein to an environment in which one or more fluids are present.

The term “biological matter” refers herein to blood, tissue, fluid, enzymes, interstitial fluid, and other secretions of a body.

The term “medicinal solution” refers herein to a preparation intended for a therapeutic, diagnostic, or other biological purpose in any form. Each medicinal solution can include one or more components, and a device or system can include one or more medicinal solutions. A component of a medicinal solution can be, for example, a pharmacological agent, a DNA or SiRNA transcript, a cell, a cytotoxic agent, a vaccine or other prophylactic agent, a nutraceutical agent, a vasodilator, a vasoconstrictor, a delivery enhancer, a delay component, an excipient, or a diagnostic agent. A component of a medicinal solution that is included in the medicinal solution for the purpose of invoking a biological effect in tissue of a body, such as any of the foregoing or other component, is referred to herein for convenience as an “active agent”.

A pharmacological agent can be, for example, an antibiotic, a nonsteroidal anti-inflammatory drug (NSAID), an angiogenesis inhibitor, a neuroprotective agent, a chemotherapeutic agent, an antibody, a nanobody, a hormone, or a biologically active variant or derivative of any of the foregoing.

A cell can be, for example, a stem cell, a red blood cell, a white blood cell, a neuron, or other viable cell. Cells can be produced by or from living organisms or contain components of living organisms. A cell can be allogeneic or autologous.

A vasodilator can be, for example, 1-arginine, sildenafil, a nitrate (e.g., nitroglycerin), or epinephrine.

A vasoconstrictor can be, for example, a stimulant, an amphetamine, an antihistamine, epinephrine, or cocaine.

A delivery enhancer can be, for example, a permeation enhancer, an enzyme blocker, an antiviral drug such as a protease inhibitor, a pH modifier, a surfactant, a fatty acid, a chelating agent, or a chitosan. A delivery enhancer can, for example, serve as a delivery medium for delivery of a component of a medicinal solution, or serve to improve absorption of a component of a medicinal solution into the body.

An excipient can be, for example, a binder, a disintegrant, a superdisintegrant, a buffering agent, an anti-oxidant, or a preservative. Excipients can provide a medium for a component of a medicinal solution (e.g., for assisting in manufacture), or to preserve integrity of a component of a medicinal solution (e.g., during manufacture, or during storage).

A diagnostic agent can be, for example, a sensing agent, a contrast agent, a radionuclide, a fluorescent substance, a luminescent substance, a radiopaque substance, or a magnetic substance.

The term “degrade” or a grammatical variation thereof (e.g., “degrading”, “degraded”, “degradable”, and “degradation”) refers herein to weakening, partially degrading, or fully degrading, such as by dissolution, chemical degradation (including biodegradation), decomposition, chemical modification, mechanical degradation, or disintegration, which encompasses also, without limitation, dissolving, crumbling, deforming, shriveling, or shrinking. The term “non-degradable” refers to an expectation that degradation will be minimal, or within a certain acceptable design percentage, for at least an expected duration in an expected environment.

The term “degradation rate” or a grammatical variation thereof (e.g., “rate of degradation”) refers herein to a rate at which a material degrades. A designed degradation rate of a material in a particular implementation can be defined by a rate at which the material is expected to degrade under expected conditions (e.g., in physiological conditions) at a target delivery site. A designed degradation time for a particular implementation can refer to a designed time to complete degradation or a designed time to a partial degradation sufficient to accomplish a design purpose (e.g., breach). Accordingly, for example, a designed degradation time can be specific to a component and/or specific to expected conditions at a target delivery site.

The terms “substantially” and “about” are used herein to describe and account for small variations. For example, when used in conjunction with a numerical value, the terms can refer to a variation in the value of less than or equal to ±10%, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%.

As used herein, a range of numbers includes any number within the range, or any sub-range if the minimum and maximum numbers in the sub-range fall within the range. Thus, for example, “<9” can refer to any number less than nine, or any sub-range of numbers where the minimum of the sub-range is greater than or equal to zero and the maximum of the sub-range is less than nine. Ratios may also be presented herein in a range format. For example, a ratio in the range of about 1 to about 200 should be understood to include the explicitly recited limits of about 1 and about 200, and also to include individual ratios such as about 2, about 35, and about 74, and sub-ranges such as about 10 to about 50, about 20 to about 100, and so forth.

The discussion now continues with respect to delivery of a medicinal solution.

There is a need for improved techniques for delivery of a medicinal solution for the treatment of brain growths and other neurological conditions. For convenience, the term “growth” as used herein encompasses tumors, cancers, and other growths to be treated (whether or not tumorous and/or cancerous). One approach is to use intracranial delivery into brain tissue or into CSF in the brain. Intracranial delivery has an advantage of delivery of an active agent directly to, or into, a growth or other targeted area, which can minimize side effects associated with other treatments (e.g., IV chemotherapy) because the intracranially delivered dose can be much lower and delivery is largely confined to the cranial cavity. Intracranial delivery poses a number of challenges, which are addressed herein. For example, one challenge is to fixate a catheter to maintain a placement of the catheter in the cranium and to minimize movement of the catheter within the cranium to prevent adverse effects on healthy brain tissue. Other challenges include preventing backflow of medicinal solution or CSF from the brain, avoiding cerebral edema from over-delivery of medicinal solution into the brain, and avoiding under-delivery of an active agent due to hindered flow of medicinal solution through the catheter.

Various embodiments provide techniques and devices for delivering a medicinal solution intracranially. Such medicinal solutions can include one or more active agents to provide targeted focal treatment of brain growths.

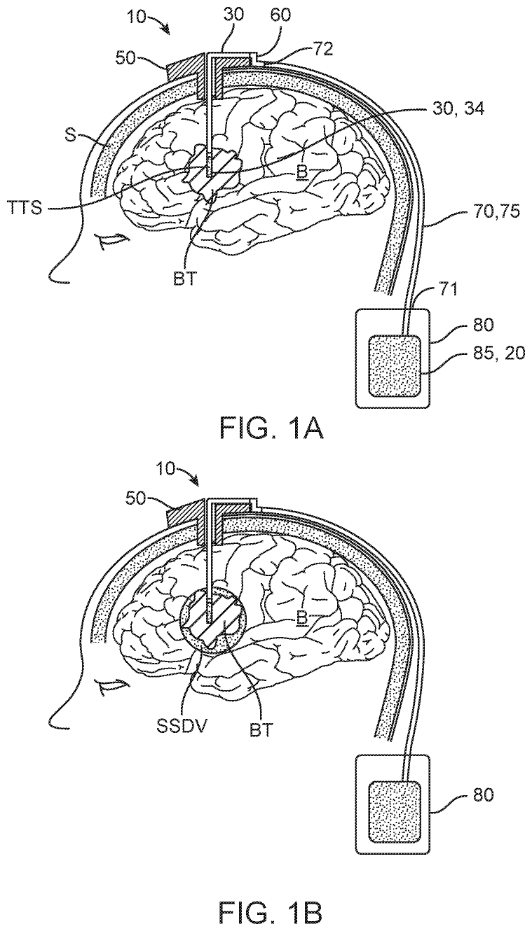

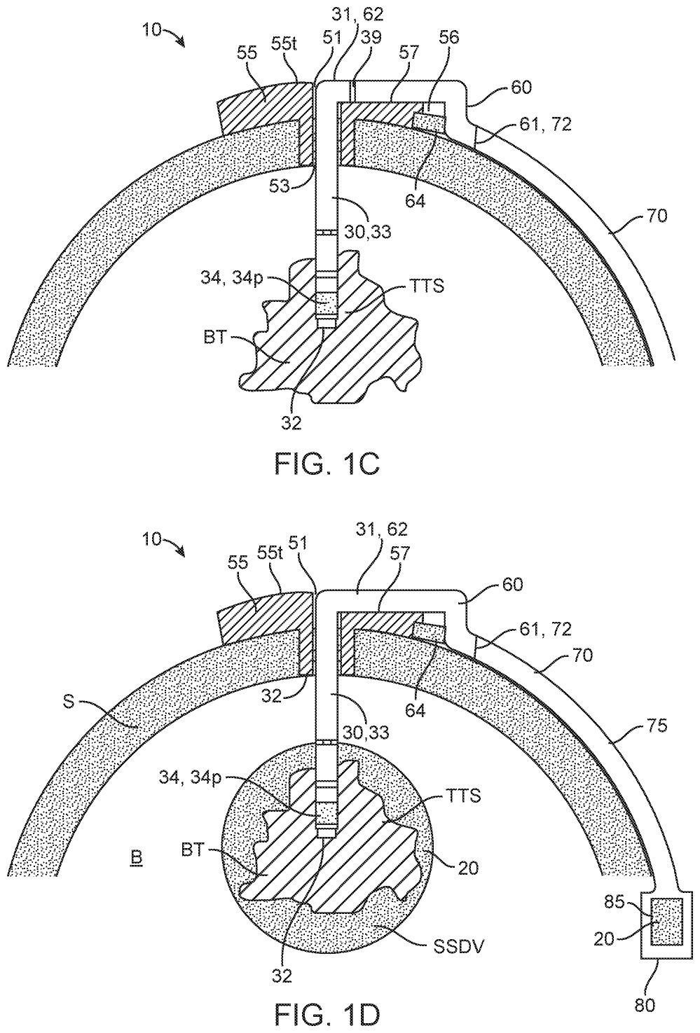

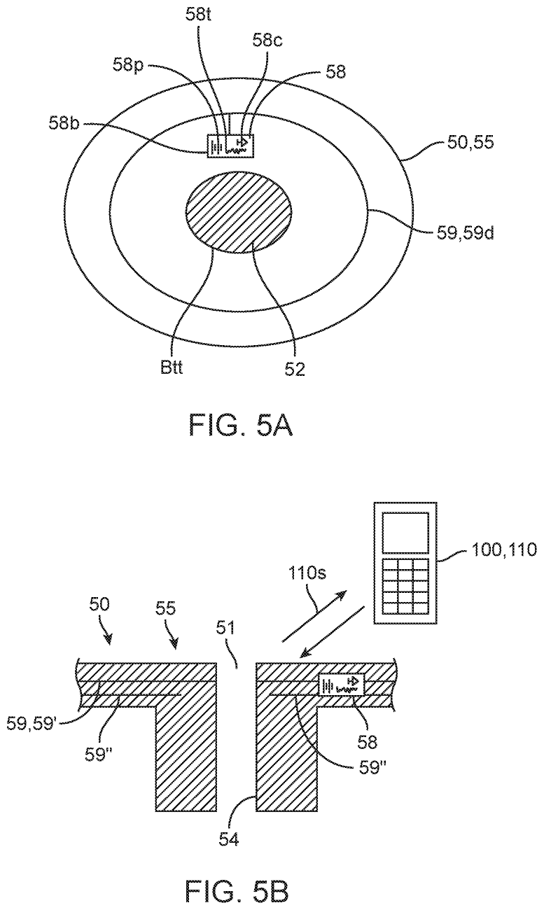

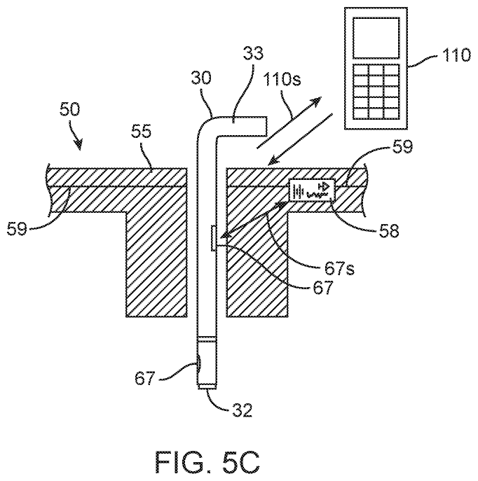

FIG. 1A-FIG. 6B illustrate embodiments of an intracranial drug delivery system 10 for intracranial delivery of a medicinal solution 20 to a target tissue site (TTS) in a brain ‘B’ of a subject. System 10 includes a catheter 30, a stylette 40 (an introducer member for introducing catheter 30 into the TTS), a cranial burr hole stopple 50 inserted into a burr hole ‘BH’ in a skull ‘S’, a connecting member 60, a connector tube 70 (a flexible tubular member), and a fluid delivery system such as a pump 80.

Pump 80 is coupled to a proximal end 71 of connector tube 70, a distal end 72 of connector tube 70 is coupled to a proximal end 61 of connecting member 60, and a distal end 62 of connecting member 60 is coupled to a proximal end 31 of catheter 30. Thus, a flow path 75 is defined from pump 80 to and through catheter 30.

Flow path 75 may include one or more lumens. Flow path 75 includes at least one fluidic lumen fluidically coupled between pump 80 and distal end 32 of catheter 30 for liquid flow (e.g., for providing medicinal solution 20) through flow path 75 to the TTS. In an embodiment, flow path 75 includes two or more fluidic lumens, each fluidically coupled between pump 80 and distal end 32 of catheter 30, where two or more of the fluidic lumens are not fluidically coupled together, such that at least two different fluids (e.g., including medicinal solution 20) can be provided intracranially (e.g., substantially concurrently, sequentially, or in an overlapping regimen).

In an embodiment, flow path 75 further includes at least one lumen for distributing one or more electrical conductors (each such lumen referred to herein as a conductor lumen), to establish electrical coupling between two or more components of system 10. For example, electrical conductors may be distributed between pump 80 and distal end 32 of catheter 30, between pump 80 and stopple 50, between pump 80 and a sensor positioned to detect a condition in or on connecting member 60 or connector tube 70, between catheter 30 and stopple 50, between stopple 50 and a sensor positioned to detect a condition in or on connecting member 60 or connector tube 70, or other electrical coupling between components. In an embodiment, flow path 75 includes at least one conductor lumen that does not extend an entirety of the length of flow path 75. For example, flow path 75 may include a first conductor lumen extending between pump 80 and stopple 50 and a second conductor lumen extending between stopple 50 and distal end 32 of catheter 30, where the first conductor lumen and the second conductor lumen do not meet.

An electrical conductor may be, for example: a wire; twisted or braided wire filaments forming a multifilament wire; a trace; or a printed conductive ink. An electrical conductor allows for energy propagation, such as in the form of delivered power or in the form of one or more signals. A signal may be transmitted on a single electrical conductor, or on multiple electrical conductors (e.g., two wires for higher current capability, two wires for signal and return, three wires for signal, return, and offset, or three wires for positive, negative, and ground). Multiple electrical conductors together may additionally or alternatively be used implement a bus, such as is used for serial or parallel communication protocols.

Where the various components defining flow path 75 are connected, various connection techniques may be incorporated, and different connection techniques may be implemented for different interfaces along flow path 75. For example, a distal end of one component may be sized to be inserted into a proximal end of another component, or a proximal end of one component may be sized to be inserted into a distal end of another component. In specific examples, a portion of catheter 30 at proximal end 31 can be sized to be inserted into connecting member 60 at distal end 62, or a portion of connecting member 60 at distal end 62 can be sized to be inserted into catheter 30 at proximal end 31. In an embodiment, one or more components defining flow path 75 include threading, ridging, flanging, or other raised sealing feature (any of which is referred to herein as a feature 38 for convenience) that is structured to engage and form a fluidic seal between the connected components. Features 38 may additionally be structured to facilitate fixation of catheter 30 to connecting member 60 to prevent an accidental dislodgement of catheter 30 from connecting member 60, such as might occur from head movement or from impact to an area of the skull ‘S’ containing stopple 50. In an embodiment, a luer-lock or other adaptor or connector is used at an interface between components.

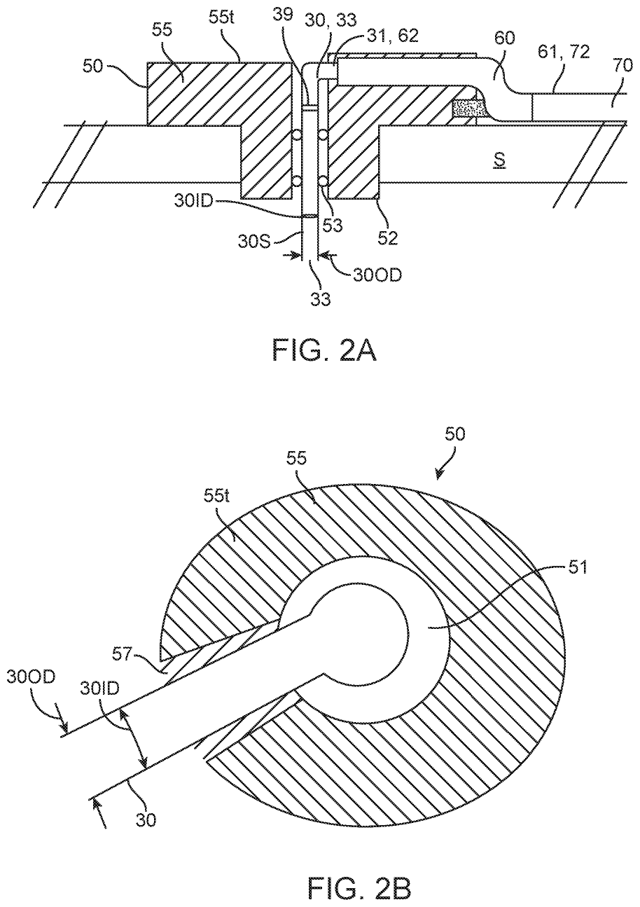

In an embodiment, one or more one-way valves 39 are disposed in flow path 75 to prevent backflow of medicinal solution 20 or CSF. For example, FIG. 1C illustrates valve 39 near distal end 62 of connecting member 60, and FIG. 2A illustrates valve 39 near proximal end 31 of catheter 30. Other positions for valve 39 are also contemplated, including but not limited to near distal end 32 of catheter 30.

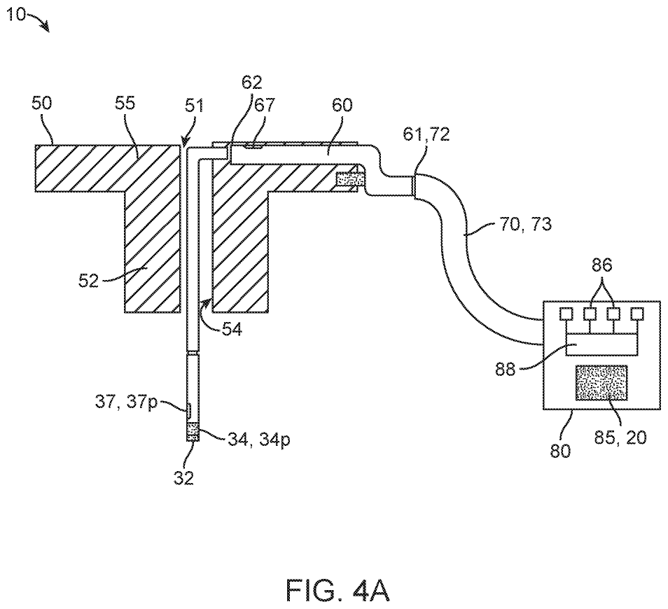

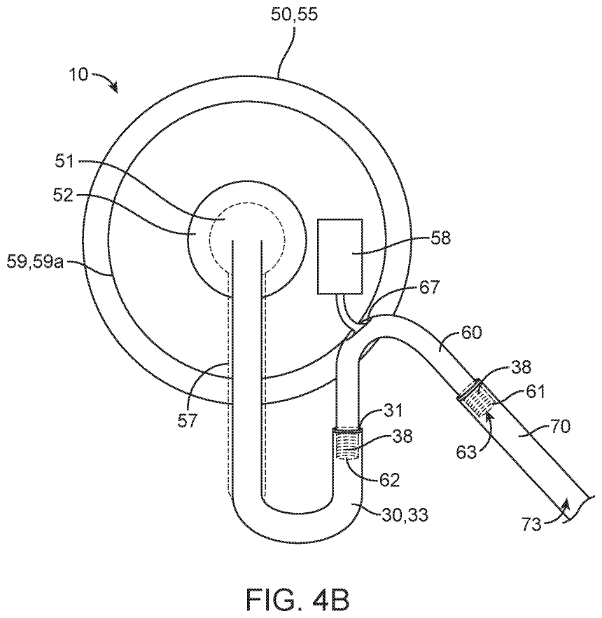

One or more sensors may be positioned at one or multiple locations in flow path 75 to detect backflow, turbulence, or reduced flow (e.g., due to a blockage). FIG. 4A illustrates an embodiment in which a sensor 67 is positioned near distal end 62 of connecting member 60. Other positions are also contemplated along flow path 75. Examples of sensors include a pressure sensor or a strain gauge.

Other sensors (such as pH or oxygen sensors) may be positioned at distal end 32 of catheter 30 to ascertain properties of tissue at or in the TTS, such as pH or level of tissue oxygenation which are indicative of cancerous tissue. Distally placed sensors within catheter 30 may, for example, measure a concentration of an active agent in tissue at the TTS to allow a medical professional or system 10 to titrate or adjust the delivery of medicinal solution 20 to the TTS.

Catheter 30 defines one or more lumens 33, including at least on fluidic lumen 33. In an embodiment, catheter 30 defines two or more fluidic lumens 33 for delivering fluid (e.g., medicinal solution 20). In an embodiment, flow path 75 includes a single lumen for delivering fluid between pump 80 and the proximal end 31 of catheter 30, and catheter 30 defines two fluidic lumens 33 to split flow path 75 into two sub-paths within catheter 30. In an embodiment, catheter 30 defines two or more lumens 33, including at least one fluidic lumen 33 and at least one conductor lumen 33, and one or more electrical conductors are disposed in one of the conductor lumens 33, such as to connect electronics at distal end 32 of catheter 30 to electronics elsewhere in system 10 (e.g., electronics of stopple 50, electronics of pump 80, or electronics external to system 10).

Medicinal solution 20 exits catheter 30 from one or more aperture(s) 34 at or near distal end 32 of catheter 30. Distal end 32 is structured to be positioned at the TTS in or near a cranial growth indicated for convenience as a brain tumor ‘BT’. Distal end 32 can include one or more features described herein such as various sensors or atraumatic structures.

In an embodiment, aperture 34 of catheter 30 corresponds to a single opening at or near distal end 32 of catheter 30. In an embodiment, aperture 34 is one of multiple apertures arranged in a pattern 34p. Pattern 34p may be, for example, a radially distributed pattern for delivery of medicinal solution 20 to a volume of brain tissue at the TTS, such as apertures 34 radially distributed at 30, 45, or 60 degree offsets to one another. In an embodiment, one or more apertures 34 in the pattern 34p can be selectively opened or closed before or after positioning in the brain to produce a selectable infusion zone at or within the TTS. Selective opening and closing can be implemented by means of a movable shutter (not shown) positioned in or on catheter 30.

Catheter 30 may include various features and components to improve ease of use, performance, reliability, and/or safety of catheter 30 and/or intracranial drug delivery system 10. For example, catheter 30 may include one or more radiopaque or other imaging markers 36 positioned at a tip portion of catheter 30 and/or at various intervals along its length to allow depth of catheter penetration in the brain to be observed using fluoroscopy or other imaging modality and thus facilitate advancement of catheter 30. Markers 36 can be positioned along catheter 30 in consistent or non-consistent intervals to allow the physician (or a robot) to assess what length of catheter has been inserted into the cranium. For example, markers 36 can be positioned at 1 cm, 2.5 cm, or 5 cm consistent intervals. For another example, a first marker 36 and a second marker 36 can be positioned a first distance from each other, a third marker 36 can be positioned a second distance from the second marker 36, a fourth marker 36 can be positioned a third distance from the third marker 36, and so forth.

Markers 36 may include radiopaque, machine visible, or otherwise detectable indicia. In an embodiment, catheter 30 is structured to be engaged by effectors of a surgical robot and can include adaptors (e.g., a proximal adaptor not shown) or other feature or element allowing for this capability.

An outer diameter 300D of catheter 30 is sized to fit through an opening 51 defined by stopple 50 while forming a fluidic seal with seals 53 in opening 51. In an embodiment, outer diameter 300D can be in a range of about 1 mm to about 5 mm, more preferably in an about 1 mm to about 2 mm range. In an embodiment, an inner diameter 301D of lumen 33 of catheter 30 can be in a range of about 0.1 mm to about 1 mm. A length of catheter 30 can vary depending on depth and location of the growth to be treated and size of the subject's head. Examples include catheter lengths in a range of about 5 cm to about 30 cm, with specific embodiments of about 10 cm, about 15 cm, about 20 cm, and about 25 cm.

In an embodiment, catheter 30 is structured to be cuttable for customizable length. For example, markers 36 may be used to allow medical personnel to cut an exposed portion of catheter 30 to a selectable length for those embodiments of catheter 30 which are cuttable. In specific embodiments, the properties, materials and structure of catheter 30 are selected to allow a physician to readily cut catheter 30 to a specific length (before or after insertion into subject brain), while leaving a clean smooth proximal end that can still form a good connection with connecting member 60, and while retaining or regaining lumen 33 of catheter 30 in an open configuration. These results can be achieved using flexible and resilient polymers such as various elastomers including silicones and polyurethanes as well as various polyethylenes (e.g., LLDPE or HDPE); the material or materials used may be cross linked (e.g., by irradiation) to increase resilience and/or strength, such as hoop resilience and/or strength, to assure that lumen 33 retains or regains an open configuration after cutting catheter 30.

In an embodiment, catheter 30 will be introduced into opening 51 of stopple 50 and advanced to the TTS by being advanced over an introducer (e.g., over a stylette 40). As such, catheter 30 is structured to have mechanical properties (e.g., pushability or flexibility) so as to be able to easily track over the introducer. In an alternative embodiment, catheter 30 is structured to be introduced and advanced to the TTS without the need for an introducer. Desirably, the structure, materials, and dimensions of catheter 30 are selected to allow it to both track over the introducer while being advanced into brain tissue, as well as bend once so positioned to minimize force imparted to surrounding brain tissue.

In an embodiment, catheter 30 is formed of a flexible material. Catheter 30 may include any number of biocompatible polymers, such as various elastomers (e.g., silicone, polyurethane, a co-polymer of silicone or polyurethane, a co-polymer of silicone and polyurethane, or PEBAX). Various super-elastic metals may be used, such as NITINOL.

Desirably, mechanical properties of catheter 30, including stiffness, are structured such that catheter 30 including its distal end 32 does not cause injury to brain tissue during advancement of catheter 30 to the TTS or afterwards. Further, desirably, catheter 30 is structured such that its advancement into the brain does not cause adverse physiological or neurologic effects such as trauma, bleeding, cerebral edema or motor or cognitive loss. This can be achieved at least in part by fabricating catheter 30 from low durometer materials (e.g., silicone or other elastomer). In an embodiment, a durometer of catheter 30 can be in a range of about 20 to about 40. In an embodiment, distal end 32 of catheter 30, or a tip portion thereof (e.g., the distal 2 cm to 3 cm of catheter 30), can be made atraumatic by being made more flexible than a remaining distal (and/or proximal) portion. For example, such tip portion of catheter 30 can have a durometer in a range of about 10 to about 20, while the remaining distal (and/or proximal) portion can have a durometer of about 20 to about 40. In an embodiment, a tip portion of catheter 30 can have an atraumatic shape, such as rounded edges or a rounded dome.

In an embodiment, lumen 33 of catheter 30 includes an inner lining of coiled wire (not shown) to maintain a patency of lumen 33 if the catheter is put into a bent or deformed position.

In an embodiment, catheter 30 is structured to be steerable, such as through use of materials which transition from a higher stiffness (less flexible) to a lower stiffness (more flexible) and back. For example, shape memory materials (e.g., NITINOL) can change shape and stiffness in response to changes in temperature. Use of such stiffness transition materials can also be employed to transition catheter 30 from a less flexible structure during placement into a more flexible structure once positioned at the TTS. In this way, catheter 30 is made more atraumatic after positioning at the TTS to reduce risk of trauma or injury to the brain tissue after placement of catheter 30.

Stylette 40 is elongated, and can have a tissue penetrating distal tip 41. Desirably, stylette 40 has sufficient stiffness to allow it to be advanced into brain tissue by manual manipulation. In an embodiment, stylette 40 includes a proximal adapter 42 to facilitate advancement by a physician or advancement by a surgical robot. Stylette 40 will generally include a resilient biocompatible metal (e.g., 304V stainless steel) or resilient polymer. Stylette 40 may include a lubricous coating 43 such as polytetrafluoroethylene (PTFE) to reduce friction with tissue as well as reducing friction with catheter 30. A length of stylette 40 can vary from about 10 cm to about 30 cm, with other lengths also considered. Stylette 40 can include one or more visual and/or radiopaque or other markings 45 for medical imaging, such as at a distal tip 41 of stylette 40 and/or at a position away from distal tip 41, and/or at other positions along stylette 40. In an embodiment, multiple markings 45 are used, and include two or more markings 45 positioned at intervals (e.g., at 1 cm, 2 cm, 2.5 cm, 5 cm, or 10 cm intervals) to allow the physician to assess how deep stylette 40 has been inserted into brain tissue as well as to do so using a medical imaging modality such as fluoroscopy. Markings 45 may also include machine visible/detectable indicia allowing for control of the advancement of stylette 40 by a surgical robot or other device. In an embodiment, stylette 40 can be structured to be engaged by effectors of a surgical robot and can include one or more adaptors (e.g., corresponding to proximal adaptor 42 or other adaptor(s)) or other feature or component allowing for this capability.

In an embodiment, stylette 40 is omitted as a component of system 10 when system 10 is commercialized as a kit because stylette 40 is separately commercially available, or omits stylette 40 because catheter 30 can be structured to be introduced and advanced to the TTS without the use of a stylette or other introducing member.

Stopple 50 can be fabricated from a rigid biocompatible material, such as a rigid polymer (e.g., high density polyethylene (HDPE), or polyetheretherketone (PEEK)), a metal (e.g., titanium), or a combination of materials. Stopple 50 includes opening 51 sized and structured for advancement of stylette 40 and catheter 30 therethrough and into the brain. Opening 51 can be centrally positioned with respect to a lengthwise axis of stopple 50, though in an embodiment, opening 51 can be positioned off-center, such as to accommodate other components positioned on or in stopple 50 (e.g., electronics or antennas). A diameter of opening 51 depends in part upon an outer diameter of catheter 30. In an embodiment, the diameter of opening 51 can be in a range of about 2 mm to about 20 mm.

In an embodiment, stopple 50 includes two portions, a plug 52 and a flange 55. Plug 52 and flange 55 may be integrally formed, or may be separate components coupled together. Flange 55 and plug 52 together define opening 51 for passage of catheter 30. Plug 52 is sized and structured to be inserted into the burr hole ‘BH’ in the skull ‘S’. Seal 53 is positioned on a wall 54 of opening 51 to form a fluidic seal with an exterior surface 30s of catheter 30. An outer diameter of plug 52 can be customized for the burr hole ‘BH’ but will generally be in a range from about 5 mm to about 20 mm, with specific embodiments of about 10 mm, about 14 mm and about 16 mm. The length of plug 52 can be in a range from about 2 mm to about 15 mm, with longer and shorter lengths contemplated.

In an embodiment, flange 55 is structured to engage and extend over an outer surface of skin over the skull ‘S’ when plug 52 is inserted into the burr hole ‘BH’. In an embodiment, flange 55 is structured to extend over the skull ‘S’ and under the skin, such that flange 55 is engaged with the skull ‘S’ while a skin flap is peeled back, and the skin flap is then positioned over flange 55.

Flange 55 defines an opening 56 on a side portion of flange 55 for insertion of an anchor 64 affixed to or incorporated with connecting member 60, and defines a groove 57 on a top portion 55t of flange 55 that is structured to engage and retain catheter 30 such that movement of catheter 30 is minimized, particularly that resulting from movement of the subject's head. In an embodiment, top portion 55t of flange 55 can include two grooves 57 so as to retain catheter 30 in at least two locations and/or at least axis and thus further reduce movement of the catheter during movement of the subject's head. Groove 57 may also be structured (e.g., through a smaller opening on the top of groove 57 relative to a diameter of catheter 30) to allow for catheter 30 to be snapped into place and held in groove 57. Further, once so positioned in groove 57, the smaller opening on top of groove 57 may also serve to protect catheter 30 from forces tending to cause compression of catheter 30 (e.g., if the head is pressed against a surface or object) including those forces which would cause compression of lumen 33 of catheter 30.

In an embodiment, flange 55 includes electronics, shown by way of example as circuitry 58, including components for powering, communicating with, and/or analyzing signals from sensors (e.g., sensor 67) associated with components of system 10. For example, circuitry 58 may correspond to one or more of a processor or other controller 58c, an RF or other transmitter 58t, or a battery or other power storage device 58p. Components of circuitry 58 may be disposed on a circuit board 58b which may be or may include a flexible circuit.

In an embodiment, circuitry 58 may be coupled to an inductive coil 59 embedded or otherwise disposed in or on top portion 55t of flange 55. Coil 59 may include various conductive metals and/or polymers. Coil 59 may be structured to be used to inductively transfer power from an inductive coil of an external device 100 for powering one or more of the electrical components associated with system 10, including sensors and/or circuitry 58. Coil 59 may also be structured as an antenna 59A for transmitting and receiving signals 110s to an external communication device 110 such as a cellular or other mobile phone using BLUETOOTH or other standard communication protocol or a proprietary protocol. In an embodiment, coil 59 may correspond to a first coil 59′ for inductive power coupling and a second coil 59″ for signal transmission (e.g., RF transmission). Signals 110s transmitted to external communication device 110 may include information from one or more sensors 67 disposed on or associated with catheter 30, connecting member 60 or other component of system 10. Such signals 110s may also include information from circuitry 58 such as a state of circuitry 58 including power levels, diagnostic checks and error conditions. Sensor 67 can also send signals 67s to circuitry 58 for receiving and processing those signals.

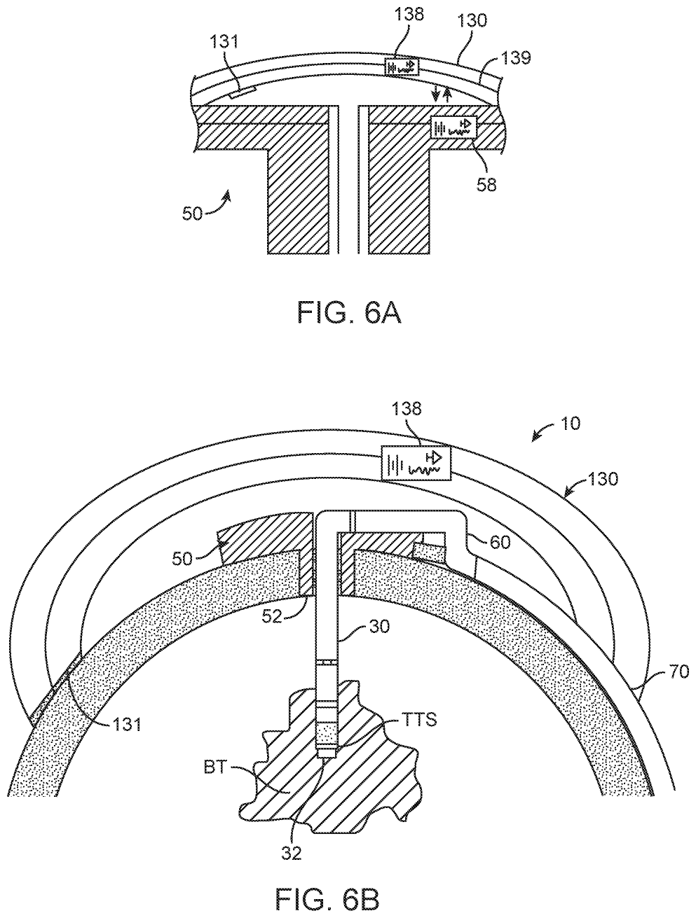

In an embodiment, external device 100 and/or external communication device 110 may correspond to or be incorporated into a head covering 130 (e.g., a skull cap or hat) worn over stopple 50 as illustrated in FIGS. 6A and 6B. Head covering 130 may include its own conductive coil(s) 139 and associated circuitry 138 for conductive power coupling and/or transmission of signals to corresponding coils in stopple 50. In an embodiment, head covering 130 includes an attachment means 131 (e.g., magnets, or hook-and-loop attachment (e.g., VELCRO)) for fixing the head covering over the area of the skull ‘S’ containing stopple 50. For magnetic attachment embodiments, portions of stopple 50, in particular, flange 55, may include ferrous materials. For hook-and-loop attachment embodiments, a flap of biomaterial (not shown) can be sutured over stopple 50, where the biomaterial includes a portion of hook-and-loop material structured to engage a portion of hook-and-loop material disposed on an inside surface of head covering 130.

Connecting member 60 defines at least one lumen 63. In an embodiment, connecting member 60 includes one or more pressure or flow sensors 67 positioned within lumen 63 (e.g., on or under a surface of lumen 63) for sensing pressure and/or flow of a fluid flowing through connecting member 60. Sensor(s) 67 can be operatively coupled to circuitry in stopple 50 or circuitry in pump 80, in either a wired or wireless fashion.

Connecting member 60 includes anchoring element 64 engaging opening 56 on the side portion of flange 55 to secure connecting member 60 to flange 55, which serves to further reduce movement of catheter 30 once positioned in the brain.

In an embodiment, connecting member 60 has a rigid elbow-like shape to retain its shape and position once attached to stopple 50. Connecting member 60 can be fabricated from biocompatible polymers (e.g., PEEK, PMMA, HDPE).

Connector tube 70 defines at least one lumen 73. In an embodiment, connector tube 70 includes one or more pressure or flow sensors 67 positioned within lumen 73 (e.g., on or under a surface of lumen 73) for sensing pressure and/or flow of a fluid flowing through connector tube 70. Sensor(s) 67 can be operatively coupled to circuitry in stopple 50 or circuitry in pump 80, in either a wired or wireless fashion.

Connector tube 70 may include one or more of various flexible polymers, including but not limited to biocompatible polymers.

Connector tube 70 may include a stiffening material or structure (e.g., a braided material) disposed in or on or forming part of connector tube 70 to avoid kinking of connector tube 70. Connector tube 70 may be of various lengths (e.g., 10 cm to 40 cm) depending on where the pump is located (e.g., where it is implanted, or how it is carried by the subject, such as implanted or carried in an area of the waist, back or pectorals) and may come in preset lengths of, for example, 10 cm, 20 cm, 30 cm, 40 cm, or other length. Connector tube 70 may be prepackaged with a kit including intracranial drug delivery system 10. Connector tube 70 may be a single segment, or may be multiple segments joined together.

Pump 80 can be selected from a variety of medical pump types. For example, pump 80 may be a displacement pump (e.g., a piston pump), a peristaltic pump, or a screw pump. Pump 80 can be miniaturized for implantation in a head or neck area of a subject (or other portion of the body). Miniaturized pumps for use as pump 80 may include MEMs and/or bubble jet based miniature pumps.

Pump 80 is also desirably programmable via means of external manual selectors (e.g., buttons or switches) operably coupled to internal circuitry 88. In an embodiment, circuitry 88 includes logic circuitry and/or a computing device such as a microprocessor, microcontroller, FPGA (field programmable gate array), PLC (programmable logic controller), or other computing device, along with associated memory and components for interfacing to the computing device.

In an embodiment, circuitry 88 may also be accessed and programmed by external communication device 110, in which case circuitry 88 includes a communication interface, either wired or wireless. Programmability of pump 80 can include, for example, allowing for control of one or more delivery parameters, such as flow rate, total volume delivered, fluid pressure, or regimen (e.g., pulsed delivery, periodic delivery, or defined on/off periods or start/stop times).

Pump 80 contains, or is structured to be coupled to, a reservoir 85 or multiple reservoirs 85. A reservoir 85 may contain a fluid, solid, or powder. In the case of a solid or powder, pump 80 includes a means to mix a liquid with the solid or powder to form a liquid solution. In an embodiment, pump 80 is coupled to an external reservoir 85 that is, or is similar to, an IV bag. Pump 80 is desirably structured to pump at low flow rates (e.g., in a 1 μl/min-50 μl/min range) and/or at low pressures.

Pump 80 desirably includes detection of: blockage in flow path 75; air in a flow path inside pump 80; a selected volume of medicinal solution 20 has been delivered; or reservoir 85 is almost empty or is empty. In an embodiment, pump 80 provides visual and/or audible alarms for one or more of the detected conditions. In an embodiment, pump 80 provides information regarding one or more of the detected conditions to external communication device 110.

In an embodiment, pump 80 is structured to be implanted in a subject (e.g., at a neck, back or pectoral area). In an embodiment, pump 80 may be worn by a subject (e.g., on a belt or shoulder strap). In an embodiment, pump 80 corresponds to a miniature infusion pump, such as a Synchromed II pump available from the Medtronic Corporation. In an embodiment, pump 80 is a miniature pump that is positioned on or adjacent to stopple 50, such as in or on top of flange 55. Desirably, such a pump has a low profile. Embodiments of such a low profile pump may include a low profile actuator which presses against or otherwise displaces a collapsible reservoir 85 to deliver fluid by displacement of reservoir 85 in response to electrical signals received by the actuator. The actuator may correspond to a piezo electric or solenoid based device (which may be MEMS based) which deforms or moves (e.g., presses against the collapsible reservoir 85) in response to electrical signals. Circuitry 88 can control one or more aspects of the delivery process, such as controlling pump 80 to deliver medicinal solution 20 to the TTS, or for controlling one or more delivery parameters. Circuitry 88 may be integral to or operatively coupled to pump 80.

Circuitry 88 can receive, analyze, and/or transmit signals received from one or more sensors 67. In addition or in the alternative, controller 58c integral to or operatively coupled to stopple 50 can receive, analyze, and transmit signals received from one or more sensors 67. Controller 58c and/or circuitry 88 can be programmed to control one or more delivery parameters, such as a regimen where medicinal solution 20 containing an active agent is delivered according to the regimen, or can be programmed to receive a signal (e.g., wireless or otherwise) to initiate delivery of medicinal solution 20 or to change the delivery regimen (e.g., from once a day to twice a day, or change a duty cycle of delivery). In this way, the delivery of medicinal solution 20 can be controlled.

Controller 58c and/or circuitry 88 can be coupled to or otherwise receive inputs from one or more pressure or flow sensors 67 positioned in or on catheter 30, connecting member 60, or other points in flow path 75 between pump 80 and catheter 30 to control delivery of medicinal solution 20 to the TTS. Controller 58c and/or circuitry 88 can also receive inputs from other sensors structured to measure tissue concentration of a delivered medicinal solution 20 or active agent contained in medicinal solution 20. These inputs can also be used to titrate the delivery of medicinal solution 20 to achieve a selected concentration of the active agent in CSF, plasma, or tissue at the TTS.

Further, sensors can be positioned on the distal end 32 or other portion(s) of catheter 30, as well as at other sites in the body (e.g., a vein or artery), to develop a pharmacokinetic model of a distribution of an active agent at multiple sites in the body. Sensors can be used to monitor systemic levels of an active agent, and information from the monitored systemic levels can be used to titrate or discontinue delivery of medicinal solution 20 when systemic concentration of the active agent reaches or exceeds a threshold level. Pump 80 may be programmed to perform the titration or discontinue pumping in such occurrence. In addition or in the alternative, the subject or medical professional or caregiver may be alerted when such a condition occurs, through an audio or visual alarm or through a message sent to or through external communication device 110.

In additional or supplemental approaches, plasma concentration of an active agent in the subject can be monitored by standard assay, such as for the first day or two after delivery of the active agent by system 10 to the TTS, and delivery can be halted or titrated based on plasma concentration.

In an embodiment, medicinal solution 20 includes topotecan, and a systemic or plasma concentration of the topotecan should be very low due to the small amounts delivered intracranially; accordingly, a threshold level of detection of topotecan systemically can be set to be very low.

Components of system 10 can be structured to be positioned in the brain under fluoroscopic, X-ray, or other imaging guidance. In an embodiment, components of system 10 are structured to be positioned in the brain using magnetic resonance imaging (MRI), and therefore components of system 10 are fabricated from materials which are MRI compatible, such as polymers and non-ferrous metals.

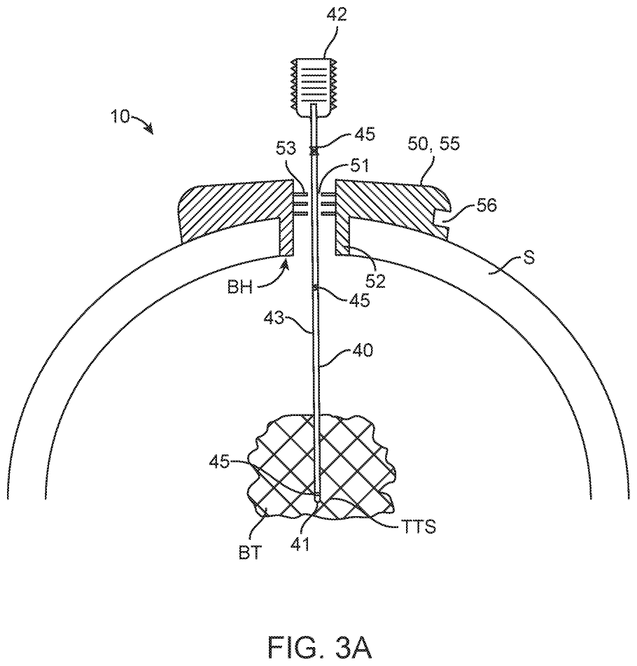

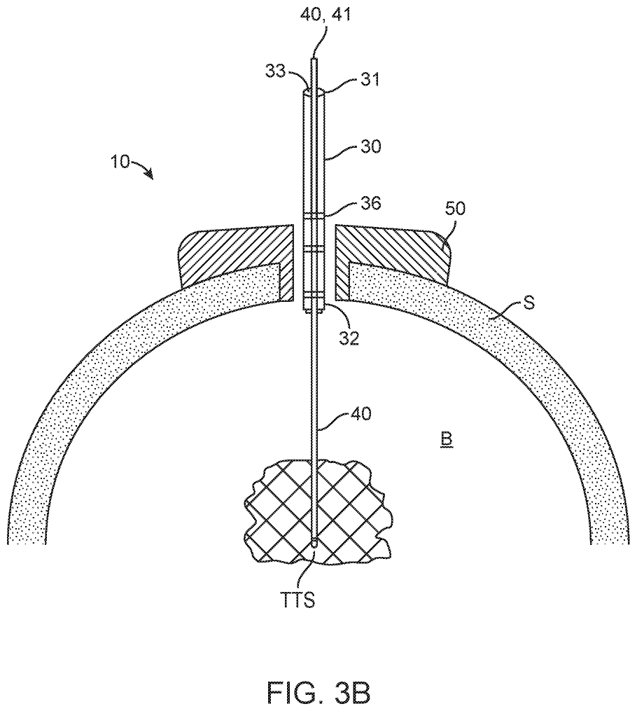

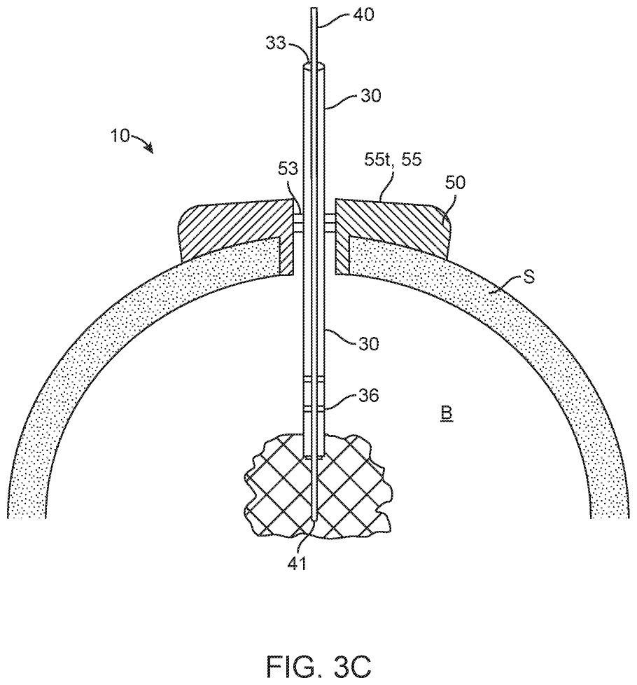

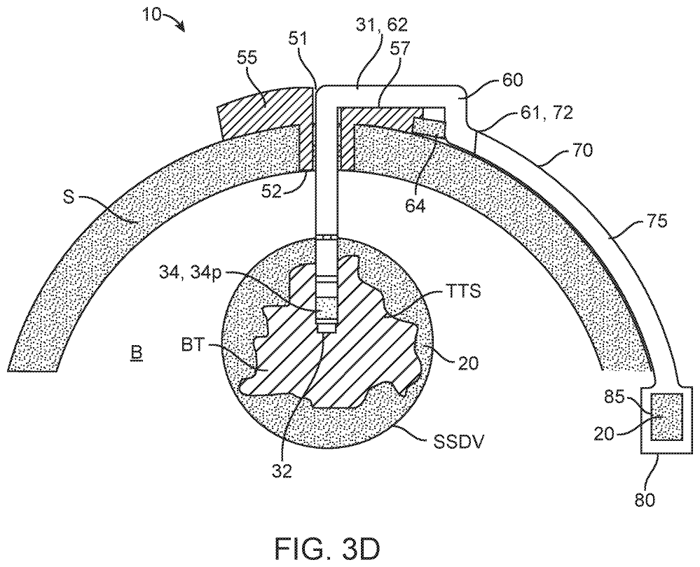

With particular reference now to FIGS. 3A-3D, an embodiment of a method for positioning and using an embodiment of system 10 for intracranial drug delivery will now be described. After imaging a subject for determination of a location and size of a selected growth ‘BT’ such as a glioblastoma, a burr hole ‘BH’ can be made in a cranium of the subject.

In FIG. 3A, the burr hole ‘BH’ is fitted with an embodiment of stopple 50. Stylette 40 is then introduced through opening 51 in stopple 50 and advanced to the TTS in or adjacent to the brain tumor ‘BT’. Advancement of stylette 40 may be done under the guidance of various medical imaging modalities which can be facilitated by a radiopaque material of stylette 40 and/or the presence of radiopaque or other imaging markers positioned on stylette 40.

In FIGS. 3B-3C, catheter 30 is advanced over stylette 40 until distal end 32 of catheter 30 is positioned at the TTS. Advancement of catheter 30 may be done under the guidance of various medical imaging modalities which can be facilitated by the presence of radiopaque or other imaging markers 36 positioned on catheter 30. Seal 53 (or multiple seals 53) can hold catheter 30 in place, and can also form a fluidic barrier in opening 51 of stopple 50. For example, seal 53 can be a septum seal or an O-ring.

In FIG. 3D stylette 40 can be removed once catheter 30 is positioned. Optionally, catheter 30 can then be cut to an appropriate length. Proximal end 31 of catheter 30 is connected to distal end 62 of connecting member 60. Before or after the connection of catheter 30 to connecting member 60, a proximal portion of catheter 30 can be positioned in one or more grooves 57 in top portion 55t of stopple 50 to fix or stabilize the exposed proximal portion of catheter 30 in one or more positions and one or more axes. Stabilization of catheter 30 serves to reduce movement of catheter 30 in brain tissue (including during head movement of the subject) and maintains distal end 32 of catheter 30 at the TTS during infusion to ensure delivery of medicinal solution 20 to the TTS.

After attachment/fixation of catheter 30 to stopple 50, connector tube 70 can be connected to connecting member 60 and to pump 80 to establish flow path 75.

In an embodiment, flange 55 of stopple 50 is sutured or otherwise affixed to skin of the subject. In an embodiment, a skin flap is sutured or otherwise affixed over a portion of stopple 50 (e.g., an exposed portion). In an embodiment, a flap of biocompatible material such as a PTFE or other membrane which serves as artificial skin is sutured or otherwise affixed over a portion of stopple 50 (e.g., an exposed portion). In an embodiment, head covering 130 is structured to engage with the skin flap, the artificial skin flap, and/or stopple 50.

Prior to or after connection of pump 80 to connector tube 70, pump 80 can be implanted at a desired tissue location in the body (e.g., in the back, base of the skull, or pectoral area of the subject), and connector tube 70 can be tunneled underneath the skin (including under skin of the scalp) with a distal portion of connector tube 70 emerging to be connected to connecting member 60 if connecting member 60 is not also disposed under the skin. Alternatively, pump 80 can be worn or otherwise carried by the subject; portions of connector tube 70 can be exposed and/or portions of connector tube 70 can be tunneled under skin and emerge close to a location where the subject will wear pump 80.

In an embodiment, reservoir 85 is preloaded with medicinal solution 20. For embodiments where reservoir 85 is implanted with pump 80, reservoir 85 may include a subcutaneous sealable access port (not shown), such as a sealable rubber septum, allowing reservoir 85 to be refilled by subcutaneous injection.

For either the implanted or non-implanted implementations of pump 80, after pump 80 is fluidically coupled to catheter 30 by means of connector tube 70, pump 80 can be turned on for a short duration to ascertain that there is no obstruction in the flow path and that medicinal solution 20 is being delivered to the TTS. In an embodiment, this process can be facilitated by an inclusion of a contrast agent mixed in with medicinal solution 20, or by having a separate reservoir (in, or coupled to, pump 80) containing contrast agent so that the TTS can be observed under fluoroscopy during pumping, to ascertain that medicinal solution 20 is reaching the TTS, and possibly also ascertain that approximately an expected amount of medicinal solution 20 is reaching the TTS. Alternatively, the physician can directly inject contrast agent into the flow path by connecting a syringe to connector tube 70 or connecting member 60 or a port coupled to either.

For embodiments of system 10 including one more pressure sensors, patency of lumen 33 of catheter 30 and delivery of medicinal solution 20 can be ascertained by pressure measurements during the test run of pump 80, with patency indicated by the pressure being within a desired range depending upon a location of the pressure sensor. After patency of lumen 33 and flow path 75 has been confirmed, and delivery of the medicinal solution 20 or contrast agent to the TTS has been established, pump 80 can be switched to a medication delivery mode (either manually at pump 80, or remotely, such as by using external communication device 110) to begin delivery of medicinal solution 20 to the TTS in the brain ‘B’.

Methods for treating a brain growth including focal treatment of the brain growth by the intracranial delivery of medicinal solution 20 using an embodiment of the intracranial drug delivery system 10 will now be described. Typically, medicinal solution 20 will include one or more active agents such as a chemotherapeutic agent which are cytotoxic to certain brain growths. Examples of chemotherapeutic agents include topoisomerase-I inhibitors such as topotecan. Medicinal solution 20 may also contain various excipients such as preservatives, or such as viscosity modifying agents (e.g., mannitol). Medicinal solution 20 may also contain an acid (e.g., hydrochloric acid in small amounts) to maintain medicinal solution 20 at an acidic pH to preserve an activity of the active agent.

After installation of system 10 or portions thereof in a body, a selected volume of medicinal solution 20 can be delivered according to delivery parameters (flow rate, total volume delivered, fluid pressure, and/or regimen) to deliver a therapeutically effective dose of an active agent in medicinal solution 20 to the TTS. After a time period of delivery of medicinal solution 20 according to the delivery parameters, one or more of growth size, rate of change of growth size, and/or other indicia of growth viability (e.g., biomarkers) can be monitored to ascertain an effectiveness of treatment, and one or more of the delivery parameters may be adjusted in response. Delivery can be adjusted relative to an original growth size, a rate of change of growth size, a change in growth size (e.g., increase or decrease), change in a biomarker (e.g., a surface antigen of the growth, DNA of the growth, or a protein produced thereby), or other indicia of the effectiveness of treatment. Growth size can be monitored by MRI, computerized tomography (CT), or computerized axial tomography (CAT) scan. Growth biomarkers can be monitored by liquid biopsy, and/or by using catheter 30 as a biopsy device by drawing a vacuum on catheter 30 using a syringe (or other vacuum source), or by structuring catheter 30 to allow for insertion of a biopsy needle (which may have a similar diameter and length as stylette 40). In similar fashion, tissue and/or fluid samples can be drawn to monitor a concentration of active agent at the TTS. Also, systemic levels of the active agent can be monitored.

In an embodiment, delivery parameters can be controlled to optimize a therapeutic effectiveness of treatment as well as minimize side effects such as toxicity to one or more organs or systems of the subject, such as to kidney, liver, or bone marrow. Toxicity to bone marrow can be in a form of bone marrow suppression, which can be determined and quantified by an occurrence of decreases in white blood cell count (in particular neutrophils) resulting in neutropenia, decreases in red blood cell count resulting in anemia, or decreases in platelets resulting in thrombocytopenia. Toxicity to the kidney and liver can be determined by monitoring for urea and liver enzyme levels. Toxicity conditions can be prevented by monitoring systemic levels of active agent delivered.

In an embodiment, a flow rate of medicinal solution 20 will be kept in a 1 μl/min-50 μl/min range to allow for long term delivery of an active agent in medicinal solution 20, and minimize a risk of cerebral edema or other adverse side effects such as allergic or other reaction. Also, when infusion of medicinal solution 20 is first started, slower flow rates can be used (e.g., 1 μl/min-2 μl/min) for the first several hours to monitor for allergic or other adverse reaction. Having observed no adverse reaction, the delivery parameters can then be adjusted to provide an increase in delivery of medicinal solution 20 the TTS. In an embodiment, delivery parameters can be adjusted by transmitting programming instructions to pump 80 from communication device 110. In an embodiment, delivery parameters can be adjusted at a manual entry system of pump 80, such as at a touch screen or through switches.

In an embodiment, a dosage of an active agent in medicinal solution 20 to be delivered is selected to produce a localized cytotoxic effect while minimizing adverse peripheral effects such as adverse effects to the kidneys (nephrosis) or liver, or bone marrow suppression that can result in hematologic effects including neutropenia, anemia and thrombocytopenia. Desirably, the delivered dosage of a particular active agent results in systemic concentrations at least 5%, and more desirably at least 10%, and even more desirably 20% or more below a threshold dosage of the active agent which produces appreciable adverse systemic effects. For example, an appreciable adverse hematologic effect can be a decrease by more than 10% in white blood cells (e.g., neutrophils), red blood cells, and/or platelets, and correspondingly, the delivered dosage of the active agent may desirably be at least 5% less than a dosage of the active agent that causes a 10% decrease in blood cells and/or platelets. For another example, adverse effects to the kidney or liver can be defined by a decrease in function of the respective organ as determined by measurement of serum creatinine or urea levels in the case of the kidney and various liver enzymes for the case of the liver; appreciable adverse effects may be considered to be more than 5% decrease in organ function, and correspondingly, the delivered dosage of the active agent may desirably be at least 20% less than a dosage of the active agent that causes a 5% decrease in organ function.

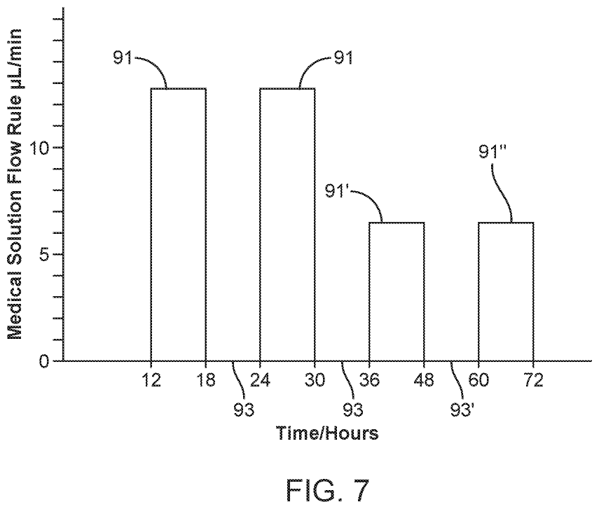

FIG. 7 illustrates an embodiment of an intracranial delivery regimen for treatment of a brain growth or other neurological condition. The regimen can include one or more “on” periods (during which medicinal solution 20 is being delivered to the TTS) and one or more “off” periods (during which medicinal solution 20 is not being delivered to the TTS). In the illustration of FIG. 7, a first and a second “on” period 91 each has a duration of about 6 hours with a subsequent “off” period 93 of about 6 hours. Thus, the regimen includes a periodicity of 12 hours with a duty cycle of 50% for the initial 36 hours, with a flow rate of approximately 12.8 μl/min. The regimen then continues with a different periodicity, of 24 hours (duty cycle 50%) and flow rate of approximately 6.5 μl/min, where a third “on” period 91′ has a duration of about 12 hours with a subsequent “off” period 93′ of about 12 hours, and a fourth “on” period 91″ has a duration of about 12 hours.

The example illustrated by FIG. 7 is provided to indicate that medicinal solution 20 can be provided for a period of time (“on” period) with a subsequent rest (“off” period), and can be provided at varying flow rates. In the embodiment of FIG. 7, the regimen includes a first periodicity (12 hours, with duty cycle 50%) and first flow rate (approximately 12.8 μl/min) and a second periodicity (24 hours, with duty cycle 50%) and second flow rate (approximately 6.5 μl/min). A regimen may include these or other periodicities, duty cycles, and flow rates, or may include continuous delivery for a single time period.

There are several benefits to an on-off treatment regimen (e.g., with a consistent or variable periodicity and/or duty cycle). First, such a regimen allows for the active agent to be delivered over a longer period of time while reducing a potential risk of toxicity to healthy brain tissue. This is because during and after infusion at the TTS, the active agent diffuses out into a diffusion volume in the brain tumor ‘BT’, which over time becomes a steady state diffusion volume (SSDV) in which a therapeutically effective concentration of active agent is maintained to act on cells of the growth. By turning off the infusion of the active agent after a set period of time, while concentration of the active agent in the growth remains at therapeutically effective levels, concentration of the active agent in the surrounding healthy brain tissue does not reach toxic levels because it is eventually flushed out by the circulation of CSF within the brain. This in turn increases efficacy of treatment (e.g., faster shrinkage of the growth and shorter times to remission) by allowing the growth tissue to be exposed to therapeutic concentrations of active agent for longer periods of time (e.g., a week or even a month) than if the infusion were done in one continuous infusion (e.g., over one day or several days). In particular, it allows for treatment of the more resistant forms including mutations of a particular type of growth over a longer period of time. For heterogeneous growths (e.g., those made up of several types of cancer cells, including those that develop by mutation), more resistant forms of cancer may not be the dominant form at first but only emerge after the less resistant more dominant type of cancer is killed off at end of a shorter course of continuous infusion. By infusing over a longer period of time using an embodiment of an on-off regimen (e.g., as illustrated in FIG. 7), a net result is that not only is the growth shrunk or put into remission faster, an incidence of reoccurrence of the cancer can be reduced, possibly significantly. This reduces the need to have subsequent treatments including the need to re-implant/re-insert one or more of catheter 20, burr hold stopple 50, or pump 50, thus reducing a risk of infection and other adverse effects associated with re-insertion and/or re-implantation of components. In various embodiments, depending upon the size and type of the growth as well as the resulting response (e.g., amount of growth shrinkage), a treatment regimen including an on-off regimen can be maintained for a period of one or more weeks, a month or even longer. Typically, the “on” and “off” periods will be in equal time duration (e.g., 50% duty cycle) or maintained in various ratios, for example a ratio of “on” to “off” periods in a range of about 4:1 to about 1:4 (e.g., duty cycle of about 25% to about 75%).

Information from a model of correlations of SSDV to one or more delivery parameters can be used to select and/or titrate a flow rate of medicinal solution 20 based on a desired SSDV. The SSDV reflects a volume of tissue in the brain having a threshold concentration of the active agent at a point in time where the inflow of medicinal solution 20 into the volume matches the outflow from the volume due to diffusion (e.g., fickian diffusion). For example, a perimeter of the SSDV may approach a spherical shape around a point of delivery. For example, a model can indicate a desired amount of active agent in the brain at steady state versus flow rate of medicinal solution 20 containing the active agent. The model can be developed using intracranial infusion of a contrast agent (e.g., iodine for X-ray/CAT scan or gadolinium for MRI) at selected flow rates (e.g., in μl/min), and the diffusion volume of the contrast agent can then be monitored by MRI or fluoroscopy until the diffusion volume reaches an SSDV.

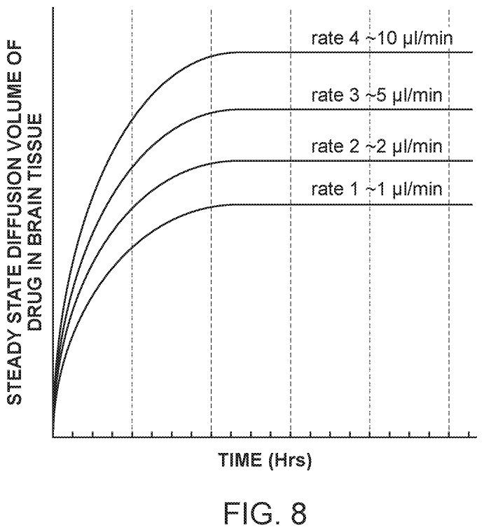

FIG. 8 illustrates an embodiment of a model to predict SSDV for a given rate of delivery of medicinal solution 20 to the TTS using embodiments of system 10. In FIG. 8, for example, diffusion volumes are plotted versus time for 1 μl/min, 2 μl/min, 5 μl/min, and 10 μl/min, showing that for each diffusion rate, an SSDV is reached (e.g., at a point in time after which the diffusion volume is approximately constant for the flow rate). The curves in FIG. 8 indicate how SSDV might vary for the different flow rates relative to each other for a particular active agent delivered.

An SSDV model can be developed based on empirical data. SSDV models can be developed using data from humans, or using data from animals whose brain anatomies approximate a human (e.g., monkey, ape, pig, or canine). An SSDV model can be updated as more data becomes available. A model based on data from one kind of animal can be updated with data from another kind of animal. A model based on non-human animal data can be updated with human data when available. Alternatively or additionally, a model based on animal data can be adjusted for humans by taking into account known or expected differences in animal versus human brains (e.g., in terms of brain volume, anatomy, or pharmacodynamic properties). An actual SSDV may have an uneven perimeter, or an asymmetrical shape around one or more axes; a shape and perimeter of each SSDV used to create a model may be manually or mathematically approximated to a standard shape and perimeter, such as approximated to a sphere with smooth perimeter. Further adjustments to the model can be made for differences in diffusion coefficients of a contrast agent (e.g., gadolinium or iodine) used in gathering data for the model versus those of a selected agent (e.g., topotecan) for which the model will be used; specific adjustments can be made based on such parameters as molecular weight, polarity, lyophilic nature, and tissue solubility of the contrast agent. Various numerical techniques can be employed to develop the model (e.g., least squares, Newton Raphson method, Euler method, Runge—Kutta, or cubit spline fit). After the model is initially developed, more data can be used adjust the model using model-fitting techniques (e.g., those incorporating an error function).

A target SSDV may be selected to be a same size or slightly larger (e.g., by several mm) than a volume occupied by a growth, to treat a selected healthy tissue margin around the growth. The SSDV may be selected depending upon the type and stage of the growth, such as smaller SSDV for initial treatment and larger SSDV for treatment of a recurrent growth, or such as larger SSDV for later-stage growth and smaller SSDV for early-stage growth, or such as larger SSDV for an initial “on” period of treatment and smaller SSDV (or trending smaller) for subsequent “on” periods of treatment. The SSDV can be selected to be smaller than the volume of the growth in some instances. The volume of the growth can be determined by one or both of a CAT scan or cranial MRI. Medicinal solution 20 may include an active agent as well as a diagnostic agent including a contrast agent to allow for visualization of the diffusion volume under fluoroscopy or MRI.

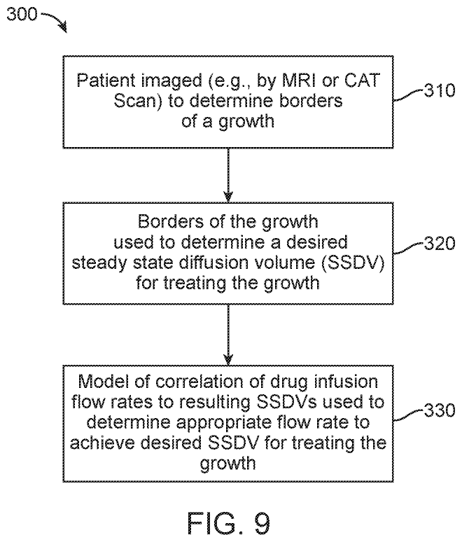

FIG. 9 illustrates embodiment of an algorithm 300 for using a model of infusion flow rate information to achieve a selected SSDV sized for treatment of a brain growth. At 310, a subject would undergo imaging such as MRI or CAT scan to determine a border of the growth. The results of the imaging may be used to determine a sphere, the volume of which would circumscribe the entirety of, or a substantial portion of, the growth. At 320, the physician would then use the determined information (e.g., shape, border, circumscribing volume) to determine a desired SSDV to treat the growth, accounting for such factors as desired healthy tissue margin. At 330, an SSDV-flow rate model is used to determine an appropriate infusion flow rate of solution (e.g., medicinal solution 20) to achieve the desired SSDV of brain tissue to be treated by an active agent in the solution.

### EXAMPLE

### Topotecan

Various embodiments of the present disclosure contemplate the intracranial delivery of a medicinal solution including topotecan. A reference to “topotecan” anywhere within the present disclosure encompasses also other analogues or derivatives of camptothecin (including water-soluble analogues), including irinotecan (and its active metabolite), 10,11-methylenedioxy-CPT (MDC), and the alkylating derivative 7-chloromethyl-10,11-MDC. A reference to “topotecan” is understood to specifically envision its analogues and derivatives.

Topotecan is a topoisomerase I inhibitor that is a semi-synthetic derivative of camptothecin. The chemical name for topotecan free base is (S)-10-[dimethylamino)methyl]-4-ethyl-4,9-dihydroxy-1Hpyrano[3′,4′:6,7] indolizino [1,2-b]quinoline-3,14-(4H,12H)-dione. It has the molecular formula C23H23N3O5 and a molecular weight of 421.45. The hydrochloride salt of topotecan is freely soluble in water and melts with decomposition at 213° C. to 218° C.

Per the FDA, topotecan solutions for injection are supplied as a sterile, non-pyrogenic, clear, light yellow to greenish solution in single-use vials at a topotecan free base concentration of 1 mg/ml. Each milliliter contains topotecan hydrochloride (equivalent to 1 mg of topotecan free base), 12 mg of mannitol (USP), and 5 mg of tartaric acid (NF), and may also contain hydrochloric acid and sodium hydroxide to adjust the pH. The solution pH ranges from 2.0 to 2.5.

Topoisomerase I relieves torsional strain in DNA by inducing reversible single strand breaks. Topotecan binds to the topoisomerase I DNA complex and prevents religation of these single strand breaks. The cytotoxicity of topotecan is thought to be due to double strand DNA damage produced during DNA synthesis, when replication enzymes interact with the ternary complex formed by topotecan, topoisomerase I, and DNA. Mammalian cells cannot efficiently repair these double strand breaks.

The dose-limiting toxicity of topotecan is leukopenia. White blood cell count decreases with increasing topotecan dose or topotecan AUC. When topotecan is administered at a dose of 1.5 mg/m²/day for 5 days by IV infusion, an 80% to 90% decrease in white blood cell count at nadir is typically observed after the first cycle of therapy.

The pharmacokinetics of topotecan have been evaluated in cancer subjects following doses of 0.5 to 1.5 mg/m² administered as a 30-minute IV infusion. Topotecan exhibits multi-exponential pharmacokinetics with a terminal half-life of 2 to 3 hours. Total exposure (AUC) is approximately dose-proportional. Distribution: binding of topotecan to plasma proteins is about 35%. Metabolism: topotecan undergoes a reversible pH dependent hydrolysis of its lactone moiety; it is the lactone form that is pharmacologically active. At pH≤4, the lactone is exclusively present, whereas the ring-opened hydroxy-acid form predominates at physiologic pH. In vitro studies in human liver microsomes indicate topotecan is metabolized to an Ndemethylated metabolite. The mean metabolite:parent AUC ratio is about 3% for total topotecan and topotecan lactone following IV administration.

Dosing for intracranial infusion of topotecan is adjusted to take into account the localized delivery directly to the TTS so that all or most of the topotecan is provided in or near the growth, as compared to the indirect route by IV with only a percentage of the topotecan reaching the growth. Intracranial infusion also avoids the systemic dispersal of topotecan through the body by IV infusion and correspondingly reduces systemic toxicity.

In an embodiment, medicinal solution 20 including topotecan for intracranial infusion can have a concentration of topotecan of 0.01 mg/ml to 1 mg/ml. In an embodiment, the concentration of topotecan in medicinal solution 20 for treatment of glioblastoma is less than or equal to about 0.1 mg/ml.

A total amount of topotecan delivered using embodiments of system 10 or other intracranial delivery systems can be in a range of about 0.1 mg to about 10 mg per treatment. In an embodiment, multiple treatments are scheduled, such as multiple treatments per day, one treatment per day, one treatment per week, or one treatment every two weeks. Each treatment may be a continuous infusion, or may be provided in on/off cycles (e.g., with a period and duty cycle) which may be static or variable (e.g., static or variable period, and/or static or variable duty cycle). The regimen may be adjusted, for example, to improve an outcome, reduce side effects, increase an SSDV, or determine a minimum effective dosing for a particular subject.

A flow rate for medicinal solution 20 can be adjusted to effect the selected regimen. For example, flow rate can be in a range of about 0.1 μl/min to about 50 l/min. A time period for a treatment depends on selected treatment type (e.g., continuous, or on/off cycles) and selected flow rate.

In one particular treatment regimen for delivery of topotecan, approximately 4 mg of topotecan can be delivered over a period of 66.7 to 333 hours corresponding to flow rates of between about 5 μl/min to about 10 l/min for a medicinal solution 20 including no more than about 0.2 mg/ml, and preferably no more than about 0.1 mg/ml, of topotecan.

Medicinal solution 20 including topotecan can include one or more excipients such as mannitol or other agent for controlling viscosity, a preservative, or an acid such as hydrochloric acid. The mannitol can range from about 1 mg/ml to about 12 mg/ml with lower amounts for lower viscosities and higher amounts for higher viscosities. In an embodiment, medicinal solution 20 containing topotecan contains little or substantially no buffering agent (e.g., less than 5% by molarity or volume, more preferably less than 1%, still more preferably less than 0.25%), and in particular little or substantially no acidic buffering agents such as tartaric acid, citric acid, malic acid, or monosodium citrate.

Such formulations of topotecan solutions with little or no buffering agent are considered contrary to teaching in the art with regard to topotecan. In particular with regard to topotecan, the art (specifically the manufacture's packaging insert per the FDA) teaches that topotecan solutions contain tartaric acid, a known acidic buffering agent. More specifically, embodiments of such formulations of topotecan solutions with little or no buffering agent are contrary to the specific teachings in the art regarding topotecan formulations for the treatment of cancer, because rather than formulate the solution including the active agent in a strong acidic buffer, embodiments of the present disclosure utilize the acidic environment of a tumor to enhance or maintain an anti-tumorigenic effect of topotecan within the confines of the tumor. Such an approach involves limiting and/or eliminating the acidic buffering capability of the formulation. Instead, embodiments of the topotecan formulations contemplated rely on the high buffering capability of CSF which bathes the entire brain tissue (which has a pH of approximately 7.28-7.32) to neutralize topotecan that leaches into healthy brain tissue. The higher pH of healthy brain tissue will rapidly inactivate the topotecan, thereby rendering it non-toxic to the healthy brain tissue. This can greatly improve the outcomes as healthy brain tissue is spared.

When infusion of medicinal solution 20 including topotecan is first started, slower flow rates can be used (e.g., 1 μl/min-2 μl/min) for the first several hours to monitor for allergic or other adverse reaction (e.g., fever). Having observed no adverse reaction, the flow rate can then be stepped up to a higher rate selected for the treatment. Such delivery regimens can be programmed into pump 80 or manually set by a medical professional. In an embodiment of treatment regimens including on-off delivery cycles, topotecan can be delivered in a regimen including an “on” period of from 6 to 12 hours and an “off” period of from 6 to 12 hours, with longer and shorter periods contemplated. This is another approach for reducing adverse reactions to medicinal solution 20 containing topotecan while allowing for a longer period of total exposure of the tumor under treatment to the therapeutically effective concentrations of topotecan.

Desirably the dosage, delivery parameters and delivery regimen for intracranial delivery of topotecan are selected to minimize or prevent any adverse reaction to the topotecan including adverse systemic or other peripheral effects. Desirably, the delivered dosage of a particular active agent results in systemic concentrations at least 5%, and more desirably at least 10%, and even more desirably 20% or more below a threshold dosage of the active agent which produces appreciable adverse systemic effects. Appreciable adverse hematologic effects include a decrease by more than 10%, more preferably by more than 5%, in one or more of the subject's white blood cells, red blood cells, or platelets. Further appreciable adverse systemic/peripheral reaction to topotecan delivery include any of the following quantitative measurements: neutrophil count of ≤1,500 cells/mm³, platelet count ≤100,000 cells/mm³, hemoglobin <9.0 gm/dl; or serum creatinine>1.5 mg/dl. These counts can be measured using CBC or other bioanalytical techniques. Adverse effects can be minimized or prevented by using embodiments of the on-off regimen described herein. In particular embodiments, adverse effects can also be minimized or prevented by delivering no more than about 10 mg, preferably no more than about 5 mg, total of topotecan or even more preferably no more than about 4 mg. Adverse effects can further be minimized or prevented by intracranial delivery at low infusion flow rates (e.g., less than 50 μl/min, more preferably less than 10 μl/min and even more preferably less than 5 μl/min at a concentration of topotecan not exceeding about 0.2 mg/ml, and preferably not exceeding about 0.1 mg/ml).

Various embodiments of the present disclosure provide devices, systems and methods for delivering medication to the brain to treat various conditions of the brain. Many embodiments provide devices, systems and methods for delivering medicinal solutions including an active agent (e.g., a chemotherapeutic or other therapeutic agent) to the brain to treat various types of brain cancer including glioblastomas. Particular embodiments provide a system and associated methods for intracranially delivering medicinal solutions including one or more active agents to targeted locations in the brain to provide targeted focal treatment of brain growths such as glioblastomas. In particular embodiments, a medicinal solution includes topotecan or other topoisomerase inhibitor (e.g., a topoisomerase I inhibitor) which has cytotoxic affects against cancer cells. An intracranial drug delivery system in accordance with the present disclosure may include a catheter or other flexible delivery member (referred to in general as a “catheter” hereinafter), a cranial access device through which the catheter is advanced into brain tissue, and a pump fluidically coupled to the catheter to pump a medicinal solution including an active agent through the catheter to a selected TTS in the brain such as the site of a brain growth. Embodiments also provide methods for using such a system to create a selected SSDV of the medicinal solution/active agent in the brain tissue. The SSDV can be matched in a selected manner to the volume of the growth. Typically, the SSDV will be selected to be somewhat larger in order to circumscribe or otherwise include a healthy tissue margin around the growth. The diffusion volume can be controlled by selection of the medicinal solution flow rate and delivery time using a predictive model which correlates these parameters to diffusion volume. Such model can be used to identify an applicable SSDV. Embodiments are particularly useful for the targeted focal treatment of brain growths such as glioblastomas which are inoperable due to their location in the brain and/or not readily treatable by radiation or related therapy. Such targeted focal therapy provides the advantage of delivering and maintaining therapeutically effective levels of active agents in the area of the brain growth for extended periods while sparing or minimally effecting surrounding healthy tissue brain tissue as well as reducing or eliminating toxicity to other tissue in the body should the active agent be delivered systemically.

In an aspect, a system for the intracranial delivery of a medicinal solution to a TTS in the brain of a subject is provided. The medicinal solution may include one or more active agents such as topotecan or other topoisomerase inhibitor as well as one more diagnostic agent including contrast media for various medical imaging modalities. In an embodiment, the system includes a catheter for infusion of the medicinal solution into the brain, a stylette or other introducer member for introducing the catheter into the TTS in the brain, a cranial burr hole stopple through which the catheter is advanced into the brain, a connecting member coupled to the catheter, a pump, and a connector tube for making a fluidic connection or flow path between the pump and the connecting member. In an embodiment, all or a portion of the aforementioned components can be fabricated from materials, including polymers and non-ferrous metals, which are MRI compatible. In particular embodiments, one or more of the stylette, burr hole stopple, catheter, connecting member, and connector tube can be fabricated from MRI compatible materials.

The introducer member will typically include an elongated stylette with a tissue penetrating end. For ease of discussion the introducer member will be referred to as a stylette but other introducing members or devices may be used instead. The stylette has sufficient stiffness allowing it to be advanced into the brain tissue by manual manipulation. In an embodiment, the stylette includes a proximal fitting or adapter. The stylette will typically include a resilient biocompatible metal (e.g., 304V stainless steel) or resilient polymer and will typically include a lubricous coating such as PTFE to reduce friction with tissue as well as with the at least one lumen of the catheter. A length of the stylette can vary from about 10 cm to about 30 cm, with other lengths also considered. The stylette also can include visual markings and/or radiopaque or other medical imaging markings at regularly spaced intervals (e.g., 1 cm, 2 cm, 2.5 cm, 5 cm, or 10 cm) to allow a medical professional to see visually see how much of the stylette has been inserted into brain tissue, as well as to do so using a medical imaging modality such as fluoroscopy. For embodiments having radiopaque markings, one or more radiopaque markers can be positioned at or near the tip of the stylette to allow the physician to visualize the location of the stylette tip in the brain under fluoroscopy. These markings may also include machine visible/detectable indicia allowing for control of the advancement of the stylette by a surgical robot or other device. Accordingly, the stylette can be structured to be engaged by effectors of a surgical robot and can include adaptors or other feature or element allowing for this capability.

The cranial burr hole stopple can be fabricated from a rigid biocompatible material including a rigid polymer (e.g., HDPE, PEEK), a metal (e.g., titanium), or a combination of both. The burr hole stopple includes an aperture or opening for advancement of the catheter into the brain. The aperture may be centrally positioned with respect to a lengthwise axis of the burr hole stopple, although the aperture can be positioned off-center instead. In an embodiment, the stopple includes two portions, a plug and a flange. The plug is sized and structured to be inserted into the burr hole and including at least one seal positioned on a wall of the plug aperture to form a fluidic seal with an exterior of the catheter. The diameter of the plug can be customized for the burr hole but typically will be in range from about 5 to 20 millimeters (mm), with specific embodiments of 10, 14 and 16 mm. The length of the plug can be in a range from 2 to 15 mm with larger and smaller lengths contemplated. The flange is structured to engage and extend over an outer surface of the subject's skull when the plug is inserted into the burr hole. The flange includes an opening of the top of the flange for passage of the catheter, an opening on a side portion of the flange for insertion of an anchoring element of the connecting member and at least one groove on a top portion of the flange structured to engage and retain the catheter such that movement of the catheter is minimized. In many embodiments, the top portion of the flange can include two grooves so as to retain the catheter in at least two positions and/or at least two axis and thus reduce movement of the catheter during head movement. In an embodiment, the flange includes circuitry. Such circuitry can include capability for powering, communicating with, and analyzing signals from sensors (e.g., pressure sensors) associated with components of the intracranial drug delivery system. In particular embodiments, the circuitry may be coupled to a least one coil embedded or otherwise disposed in or on a top portion of the flange. The coil(s) may be structured to be used to inductively transfer power from an external coil for powering one or more of the electrical components or sensors. The coil(s) may be structured as an antenna for transmitting and receiving signals to and from an external communication device such as a cell phone using BLUETOOTH or other communication protocol. In an embodiment, the least one coil may correspond to a first coil for inductive power coupling and a second coil for signal transmission (e.g., RF transmission). In an embodiment, the at least one coil is structured both as an antenna and as a power transfer coil. The signals transmitted to the external device may include information received from one or more sensors disposed on or associated with a component of the intracranial drug delivery system. Such signals may be transmitted from the circuitry (embedded or otherwise contained) in the burr hole stopple. Other information transmitted by way of the antenna may include information on the state of the circuitry including power levels, diagnostic checks and error conditions. In an embodiment, the external communication device may correspond to a head covering (e.g., a skull cap or hat) worn over the burr hole stopple. The head covering may include its own conductive coil(s) and associated circuitry for conductive power coupling and/or transmission of signals to corresponding coils in the burr hole stopple. In an embodiment, the head covering may include magnetic or other attachment means (e.g., hook-and-loop for fixing the head covering over the area of the skull containing the burr hole stopple. For the magnetic attachment embodiments, portions of the burr hole stopple may include ferrous based materials. For hook-and-loop attachment embodiments, a flap of biomaterial suture over the burr hole stopple may include a portion of hook-and-loop material structured to engage a portion of hook-and-loop material disposed on an inside surface of the head covering.

The connecting member includes a proximal and distal end with the distal end being structured to be coupled to the proximal end of the catheter and the proximal end to the connector tube. In an embodiment the proximal and distal end may include a luer-lock or other connector structured to connect to corresponding connectors on the proximal end of the catheter or the distal end of the connector tube. The connecting member also includes a lumen in fluidic communication with the at least one lumen of the catheter for flow of the medicinal solution; and an anchoring element engaging the opening on the flange side portion so as to secure the connecting member to the flange. The connecting member can have a rigid elbow—like shape. The connecting member can be fabricated from biocompatible rigid polymers (e.g., PEEK, PMMA, HDPE) so that it will retain its shape and position once attached to the burr hole stopple. It may also include one or more pressure or flow sensors positioned on or underneath an inner surface of the connecting member lumen for sensing pressure and or flow of a medicinal solution or other solution flowing through the connecting member. The sensor can be operatively coupled to the circuitry in the burr hole stopple (typically in the flange) either wirelessly or using wires for unidirectional or bidirectional communication between the sensor and the circuitry.

The connector tube may include various flexible polymers and includes a proximal and distal end with the proximal end coupled to the pump or other fluid delivery means and the distal end coupled to the connecting member proximal end so as to create a flow path between the pump and the connecting member and ultimately including the catheter. The connector tube may include a stiffening braid disposed in the tubing wall or on the tubing external surface so as to keep the tubing from kinking. The connector tube may be of various lengths (e.g., 10 cm to 40 cm) depending upon where the pump is implanted or carried by the subject, such as at the waist or in the back or pectoral area, and may come in preset lengths of 10, 20, 30 40 or other length that is prepackaged with a kit including or more components of the intracranial drug delivery system. The connector tube may also include various medical connectors including luer lock connectors to connect to corresponding connectors on one or both of the pump and connecting member.

A catheter includes at least one fluidic lumen for the delivery of the medical solution to the TTS with at least one aperture for exit of the solution positioned at or near the distal end or tip of the catheter. The catheter distal end is structured to be positioned at the TTS and can include one or more features such as various sensors or atraumatic structures. The proximal end is structured to be coupled to the distal end of the connecting member so as to form a flow path between the connecting member lumen and the least one fluidic lumen. This can be accomplished by sizing the two members such that the catheter can be inserted into the connector member or vice versa. To facilitate a good fluidic seal between the catheter and the connecting member, the proximal end of the catheter can include ridges or other raised sealing feature structured to engage and form a fluidic seal with the inside surface of the connecting member lumen when the catheter proximal end is inserted into the distal end of the connecting member lumen. The ridges or other raised sealing feature may also be structured to fix the catheter proximal end to the connecting member to prevent the accidental dislodgement of the catheter from the connecting member such as might occur from head movement or impact to the area of the skull containing the burr hole stopple. In an embodiment, the proximal end of the catheter can include a luer lock or other medical connector structured to connect with a corresponding connector at the distal end of the connector member.

In an embodiment the least one aperture of the catheter may correspond to a single opening placed at or near the distal end of the fluidic lumen. In an embodiment, the at least one aperture may include a plurality of apertures which may be arranged in a pattern (e.g., a radially distributed pattern) for delivery of the medicinal solution to a wider or larger volume of brain tissue at the TTS. In an embodiment, the apertures can be radially distributed at 30, 45, or 60 degree offsets to one another. One or more of the apertures can be selectively opened or closed before or after positioning in the brain in order to produce a selectable infusion zone within the TTS. Such embodiments can be implemented by means of a movable shutter positioned within the internal fluidic lumen wall. The shutter can be manually moved by prior to implantation or afterwards by means of magnetics or piezo electric materials. In an embodiment, control of the apertures can be achieved through the use of shape memory materials which change shape (e.g., diameter in response to change in temperature or other condition).

The outer diameter of the catheter can be in a range of about 1 mm to about 5 mm, more preferably 1 to 2 mm, while the inner diameter (e.g., the diameter of the lumen) can be in the range of about 0.2 mm to about 1 mm. The length of the catheter can vary in, such as within a range of 5 cm to 30 cm, with specific embodiments of 10 cm, 15 cm, 20 cm, and 25 cm. The outer portions of the catheter can include visible markers positioned at 1 cm, 2.5 cm, 5 cm or other intervals along the length of the catheter to let the physician know what length of catheter has been inserted into the subject's brain tissue. They may also be used to allow the physician to more easily cut an exposed proximal portion of the catheter to a selectable length for those embodiments of the catheter which are cuttable. These marking may also include machine visible or otherwise detectable indicia allowing for control of the advancement of the catheter by a surgical robot or other device. In these and related embodiments, the catheter can be structured to be engaged by effectors of a surgical robot and can include adaptors or other feature or element allowing for this capability.

The catheter diameter is sized to be able to fit through the aperture of the burr hole stopple while forming a fluidic seal with the one or more seals in the aperture of the burr hole stopple. Typically, the catheter will be introduced into the aperture of the burr hole stopple and advanced into brain tissue to the TTS by being advanced over a flexible introducer stylette using the fluidic lumen or in some cases a separate lumen. As such, the catheter is structured to have mechanical properties (e.g., pushability or flexibility) so as to be able to easily track over the stylette. In alternative embodiments, the catheter can be structured to be introduced and advanced to the TTS without the need for the introducer stylette. Such function can be achieved using various catheter structural components and fabrication techniques, for example through the use of a reinforcing braid or stiffening wire.

The catheter may include any number of flexible biocompatible polymers including, for example, various elastomers such as silicones and polyurethanes and co-polymers thereof and PEBAX. Other embodiments may employ various super-elastic metals, such as NITINOL. In specific embodiments, the materials, properties and structure of the catheter are selected to allow the the catheter to be readily cut to a specific length (before or after insertion into the brain) while leaving a clean smooth proximal end that can still form a good connection with the distal end of the connecting member. Also, desirably the least one fluidic lumen of the catheter stays patent after cutting. These results can be achieved by the use of flexible and resilient polymers including various elastomers including silicones and polyurethanes as well as various polyethylenes (e.g., LLDPE or HDPE) which may be cross linked by irradiation to increase their resilience and in particular their hoop resilience/strength to assure the lumen stays patent after cutting.

Desirably, mechanical properties of the catheter, including stiffness, are structured such the catheter including the tip does not cause injury to the brain tissue during advancement of the catheter to the TTS or afterwards. Further, desirably the catheter is structured such that its advancement into the brain does not cause adverse physiological or neurologic effects such as trauma, bleeding, cerebral edema or motor or cognitive loss. This can be achieved by fabricating the catheter from low durometer materials (e.g., silicone or other elastomer). For example, a durometer of the catheter can be in a range of 20 to 40. A distal portion of the catheter, for example, a tip portion (e.g., the distal 2 cm to 3 cm of the catheter) can be made atraumatic by being made more flexible than the remaining proximal portion. For example, the tip or other distal portion of the catheter can have a durometer in the range 10 to 20 while the remaining proximal portion can have durometer of 20 to 40. Also, the tip portion can have an atraumatic shape (e.g., rounded edges).

The catheter may include various features and elements to improve one or more of the ease of use, performance, reliability, and safety of the catheter and/or the intracranial drug delivery system in general. For example, the catheter may include one or more radiopaque or other imaging markers positioned at the tip as well as at various intervals in order to allow the depth of catheter penetration in the brain to be observed using fluoroscopy or other imaging modality and thus facilitate advancement of the catheter. The at least one lumen of the catheter may also include an inner lining of coiled wire to maintain the patency of the lumen if the catheter is put into a bent or deformed position. Further, in an embodiment the catheter (or the connecting member) may include a one way valve so as to prevent backflow of medicinal solution or CSF out of the catheter. The catheter may also include one or more sensors to perform one or more functions. For example the catheter may include one or more pressure sensors in order to sense the pressure as well as the flow of medicinal solution moving through or out of the catheter. Such pressure sensors may be disposed on an inside surface of the fluidic lumen or beneath it. They may also be positioned within a proximal and/or distal portion of the fluidic lumen or other location so as to measure pressure at multiple locations in the catheter lumen and ascertain that solution is flowing through the lumen and out of the catheter as well as ascertain any blockages within the fluidic lumen and their location. The pressure sensors may correspond to a MEMS or other miniature based pressure sensor such as a MEMS based strain gauge or other sensor. Other sensors (such as pH or oxygen sensors) may also be positioned at the distal end or other distal portion of the catheter to ascertain properties of tissue in the TTS such as pH or level of tissue oxygenation which are indicative of cancerous tissue. Distally placed sensors on the catheter may correspond to sensors for measuring concentration of an active agent (e.g., topotecan) in tissue at the TTS in order to titrate or adjust the delivery of medicinal solution to the TTS. In use, such concentration sensors allow for the more precise maintenance of therapeutically effective levels of active agent in the TTS. Again, one or more of the aforementioned sensors may correspond to MEMs based sensors allowing them to be easily fit on or into the catheter tip or lumen surface. They may also be operatively coupled to electronics in the burr hole stopple by means of wires or wirelessly through the use of RF ID type chips coupled or incorporated into the sensors.

In additional or alternative embodiments, the catheter can be structured to be steerable using catheter technology. In particular embodiments, this can be accomplished through the use of materials which transition from a higher stiffness (less flexible) to a lower stiffness (more flexible) and back. In particular embodiments this can be achieved through the use shape memory materials which change shape and stiffness in response to changes in temperature (NITINOL being an example). Use of such stiffness transition materials can also be employed to transition the catheter into a more flexible structure once the catheter tip is positioned at the TTS. In this way, the catheter is made more atraumatic after positioning at the TTS to reduce a risk of trauma or injury to brain tissue after catheter placement.

The pump may correspond, for example, to a displacement pump (e.g., a piston pump), a peristaltic pump, or a screw pump. The pump is also desirably programmable via means of external buttons/switches or an external communication device such as a cell phone. The programmability capability allows for control of one or more of flow rate, total volume delivered, regimen, and pump pressure. The pump contains or is structured to be coupled to a reservoir which may correspond to an IV bag or other reservoir. The pump desirably is structured to pump at low flow rates (e.g., in the 1-50 μl/min range, or even in the 1-10 μl/min range) and at low pressures. The pump also desirably can detect and signal alarms when it detects one or more of the following: blockages in the flow path, air in the flow path inside the pump, when a selected volume of solution has been delivered, or when the IV bag or other reservoir is almost empty or is empty. In an embodiment, the pump is structure to be implanted (e.g., at the base of the neck, or in the back or pectoral area). In an embodiment, the pump may be worn by the subject (e.g., on a belt or shoulder strap). In particular embodiments, the pump corresponds to a miniature infusion pump allowing for the pump to be readily implantable or easily worn by the subject. In alternative embodiments, the pump may correspond to a miniature pump that is positioned on or adjacent the burr hole stopple, such as in or on top of the burr hole stopple flange. Desirably such a pump has a low profile in order to fit over, on, or in the flange. Embodiments of such a low profile pump may include a low profile actuator which presses against or otherwise displaces a collapsible medicinal solution reservoir to deliver the fluid by displacement of the reservoir in response to electrical signals received by the actuator. The actuator may correspond to a piezo electric or solenoid based device which deforms or moves (e.g., presses against) the collapsible reservoir in response to electrical signals.

The intracranial drug delivery system can include or be operably coupled to at least one controller for controlling one or more aspects of the medication delivery process including, for example, control of the pump for delivering the medicinal solution to the TTS. In an embodiment, the controller may be integral to or operatively coupled to the pump for controlling one or more of the flow rate, pressure, total volume to deliver, or regimen of medicinal solution infused into the TTS. In additional or alternative embodiments, the system may include a second controller integral to or operatively coupled to the burr hole stopple for receiving, analyzing and transmitting signals received from one or more sensors disposed in the catheter or connecting member. The controller(s) may correspond to a microprocessor or other logic device which can be programmed to include a delivery regimen wherein medicinal solution or other medication is delivered at regular intervals (e.g., once or twice a day) over an extended period. It can also be structured to receive a signal (e.g., wireless or otherwise) to initiate the delivery of medication or to change the delivery regimen (e.g., from once a day to twice a day). In this way, the subject or a medical care provider can control the delivery of medicinal solution.

The controller can be coupled to or otherwise receive inputs from pressure or flow sensors positioned in the catheter, connecting member or other points in the flow path between the pump and the TTS as to control delivery of medicinal solution to the TTS. The controller can also receive inputs from other sensors structured to measure the tissue concentration of the delivered active agent. These inputs can also be used to titrate the delivery of the medicinal solution to achieve a selected concentration of active agent (e.g., in CSF, plasma, or tissue). Such sensors can be positioned on the distal tip or other distal portion of the catheter as well as other sites in the body (e.g., a vein or artery), in order to develop a pharmacokinetic model of the distribution of the active agent at multiple sites in the body.

An embodiment of a method for positioning and using a system and its components for intracranial drug delivery will now be described. After imaging for determination of the location and size of a selected growth (e.g., brain tumor such as a glioblastoma), a burr hole can be made at the top or other portion of the subject's cranium using surgical techniques and can be fitted with an embodiment of the burr hole adapter described herein. The stylette can then be introduced through the opening in the burr hole stopple and advanced to the TTS in or adjacent the growth under medical image guidance (e.g., fluoroscopy). The catheter is then advanced over the stylette until the distal tip is positioned at the TTS. Again advancement may be done under the guidance of various medical imaging modalities which can be facilitated by the presence of radiopaque or other imaging markers positioned at the tip as well as at various intervals along the length of the catheter. After removal of the stylette, the catheter can be held in place through the presence of seals, such as a septum-like seal or an O-ring positioned within the aperture or opening of the burr hole stopple. While the infused catheter remains fixed, depending upon the depth of insertion the physician can then cut the proximal end of the catheter and attach it to the distal end of the connecting member. Before or after the attachment of the catheter to the connecting member, the physician can insert and fix a proximal portion of the catheter in one or more grooves on the top surface of the burr hole stopple so as to fix or stabilize the exposed proximal portion of the catheter in one or more axis. In use, such techniques and structures of the system for stabilization of the catheter serve to reduce movement of the catheter in brain tissue (including during head movement of the subject head) and maintain the distal end of the catheter at the TTS during infusion to ensure delivery of the medicinal solution to the TTS.

After attachment/fixation of the catheter to the burr hole stopple the physician can then connect the connector tube to the connecting member and the pump to create a flow path between the pump and the connecting member and ultimately to and through the catheter. The physician can then suture a skin flap over the exposed portion of the burr hole stopple, or alternatively can suture or otherwise fix a flap of biocompatible material such as a PTFE or other membrane which serves as artificial skin and covering for the burr hole stopple. The burr hole stopple covering can be structured to engage with another head covering which includes at least one conductive coil for inductively powering and/or communicating with electronic circuitry contained in the burr hole stopple cover and/or the catheter. After connection of the pump to the flow path (and in some case before connection), the pump including the reservoir can be implanted at a desired tissue location such as in the subject's back, the base of the skull, or the pectoral area. The connector tube can be tunneled underneath the skin, including under the skin of the subject's scalp with the distal portion of the connector tube emerging to be connected to the connecting member. Alternatively, the pump can be worn or otherwise carried by the subject (for example around their waist by means of a belt or a clip for a belt), and a portion of the connector tube (if implanted) can emerge close to the location where the subject will wear the pump (e.g., around their waist). The reservoir can be preloaded with a solution (e.g., a medicinal solution). For embodiments where the pump is implanted with the reservoir, the reservoir may include a subcutaneous sealable access port, such as a sealable rubber septum allowing the reservoir to be refilled by subcutaneous injection. For either the implanted or non-implanted implementations of the pump, after the pump is fluidically coupled to the catheter by means of the flow path, the pump can be turned on for a short duration (and/or be programed to do so) to ascertain that there is no obstruction in the flow path and that the medicinal solution is being delivered to the TTS. In an embodiment, this process can be facilitated by the inclusion of contrast agents mixed in with the medicinal solution, or by having a separate reservoir (with or coupled to the pump) containing contrast agent so that the physician can observe the TTS under fluoroscopy during pumping to ascertain that the medicinal solution is reaching the TTS. Alternatively, contrast agent can be directly injected into the flow path by connecting a syringe to a port coupled to the flow path. For embodiments of the system including one or more pressure sensors, patency of the flow path and delivery of the medicinal solution can be ascertained by pressure measurements during the test run of the pump with patency indicated by the pressure being within a desired range depending upon the location of the pressure sensor in the catheter. For example, for proximal placement of the sensor, the pressure being too high may indicate a blockage in the flow path, whereas for distal placement of the sensor, blockage of the flow path may be indicated by the pressure being too low (e.g., due to blockage in a proximal portion of the catheter lumen or other portion of the flow path). After patency of the flow path and delivery of the medicinal solution/contrast agent to the TTS have been determined, the pump can be switched to a medication delivery mode (either manually, or wirelessly such as using an external communication device) to begin delivery of the medication solution including one or more active agents such as topotecan to the TTS.

In another aspect, a method of treating a brain tumor includes focal treatment of the brain tumor by the intracranial delivery of a medicinal solution using an embodiment of the intracranial drug delivery system described above. Typically, the medicinal solution will include one or more active agents which are cytotoxic to brain tumors such as glioblastoma, with other agents also contemplated, both therapeutic and diagnostic. For example, an active agent may be a chemotherapeutic agent. Chemotherapeutic agents include topoisomerase-I inhibitors such as topotecan. The medicinal solution may also contain various excipients including preservatives, viscosity modifying agents such as mannitol, and contrast agents to ascertain delivery of the solution to the TTS. The medicinal solution may also contain an acid (e.g., hydrochloric acid in small amounts) so as to maintain the solution at an acidic pH to preserve the activity of the active agent (e.g., topotecan). After installation of the delivery system, a selected volume of medicinal solution can then be delivered at selected flow rates over a selected time period so as to deliver a therapeutically effective dose of the active agent to the TTS. After delivery of an active agent, one or more of tumor size (and rate of its change) and/or other indicia of tumor viability (e.g., biomarkers) can be monitored so as to ascertain the effectiveness of treatment and one or more delivery parameters can be adjusted accordingly. Tumor size can be monitored by MRI or CAT scan while tumor biomarkers can be monitored using liquid biopsy techniques and/or by using the catheter as a biopsy device by drawing a vacuum on the catheter using a syringe (or the vacuum source) or by structuring the catheter to allow for the insertion of biopsy needle which may have a similar diameter and length as the stylette. The same procedure can be used to draw tissue and/or fluid samples to monitor the concentration of the active agent at the TTS. Systemic levels of the active agent can be monitored as well.

In various embodiments, the flow rate, delivery regimen and other parameters of the delivery can be controlled to optimize the therapeutic effectiveness of treatment of the active agent(s) as well as minimize side effects in particular toxicity to one or more organ/systems such as bone marrow, kidney, or liver. Delivery parameters can also be adjusted relative to the original tumor size and rate of growth of the tumor and/or changes in tumor size (e.g., shrinkage) or a biomarker (e.g., a surface antigen of the tumor, DNA of the tumor, or a protein produced thereby) or other indicia of the effectiveness of treatment. Typically, the flow rate of the medicinal solution will be kept in the 1 μl/min-50 μl/min range so as to allow for long term delivery of an active agent and minimize the risk of cerebral edema or other adverse side effects such as allergic or other related reaction. Also, when infusion of the medicinal solution including the selected active agent (e.g., topotecan) is first started, slower flow rates can be used (e.g., 1 μl/min-2 μl/min) for the first several hours to monitor for allergic or other adverse reaction (e.g., fever). Having observed no adverse reactions, the flow rate can then be stepped up to a higher rate. Such delivery regimens can be programmed into pump 80 or manually set. For the case of topotecan, the flow rate can be in the range of 1 μl/min-50 μl/min, more particularly in range of 1 μl/min-10 μl/min while the total delivery period can be in range of 12 to 100 hours, with particular embodiments of 24, 36, 48, 60, 66.7, 72, 84, and 96 hours. In one particular treatment for delivery of topotecan, approximately 4 mg of topotecan can be delivered over a period of 66.7 to 333 hours corresponding to flow rates of between 10 μl/min to 5 μl/min for a medicinal solution including about no more than about 0.2 mg/ml preferably 0.1 mg/ml of topotecan.

A method for an intracranial drug delivery regimen for treatment of a growth or other neurological conditions includes a regimen of one or more on and off periods of infusion of a medicinal solution including an active agent. Examples of on and off periods (that is, the period of time that infusion is either on or off) may be in the range of about 2 to 24 hours with particular embodiments of 4, 6, 8, 10, 12, 14 16, 18, and 20 hours.

In additional embodiments for methods of focally treating a growth by intracranial delivery of a medicinal solution using embodiments of the described intracranial drug delivery system, information from a model of correlations of SSDV of a medicinal solution in the brain vs flow rate of the solution can be used to select and/or titrate a flow rate of the medicinal solution based on a desired SSDV of medicinal solution in the brain. The SSDV being the volume of tissue in the brain having a threshold concentration of medicinal solution/therapeutic agent at a point in time where the inflow of medicinal solution into the volume from infusion matches the outflow from the volume due to diffusion (e.g., fickian diffusion). The correlations can be developed using intracranial infusion of a contrast agent (e.g., iodine for X-ray or gadolinium for MRI) in an animal model using embodiments of the intracranial drug delivery system described herein at selected flow rates and the diffusion volume of the contrast agent can then monitored under MRI or fluoroscopy until it reaches an SSDV. Adjustments can also be made for differences in diffusion coefficients of the contrast agent vs the selected active agent (e.g., topotecan).

Typically, the SSDV will be selected to be the same size or slightly larger (e.g., by several millimeters) than the subject's brain growth volume in order to treat a selected healthy tissue margin around the growth depending upon the type and stage of the growth. However, the SSDV can be selected to be larger (by more than several millimeters) or smaller than the growth volume, again depending upon the type and stage of the growth. The growth volume can be determined by one or both of a CAT scan or cranial MRI. The medicinal solution may include one or more active agents, one or more diagnostic agents (e.g., contrast agent/media to allow for visualization of the diffusion volume under fluoroscopy (using an iodine based agent) or MRI (using gadolinium based contrast agent). In an embodiment of such a method, the subject would undergo imaging such as MRI or CAT scan to determine the growth volume if possible the type of growth. The physician would then use that information, in particular the growth volume, to determine an appropriate infusion flow rate of the medicinal solution to achieve a desired treatment volume of brain tissue to be treated by the medicinal solution. The treatment volume being selected to be somewhat larger than growth volume in order to treat a healthy tissue margin surrounding the growth. The determination would be made by correlating the desired treatment volume to a database of treatment volumes achieved by selected flow rates of medicinal solution.

In yet another aspect, a method for focally treating a brain growth includes intracranially delivering to a target tissue in the brain within or in proximity to the growth, a medicinal solution including an active agent (e.g., chemotherapeutic agent) which is degraded or chemically altered at a pH at or above that found in healthy brain tissue, wherein the solution has substantially no buffering agent including, for example no acidic buffering agents. Such acidic buffering agents may include one or more of tartaric acid, citric acid, malic acid or monosodium citrate. For example, the active agent has a cytotoxic effect on cancerous tissue in a tumor but is at least partially deactivated or otherwise loses some measure of therapeutic effect against cancer cells upon contact with or after entering into healthy brain tissue surrounding the tumor by the normal physiologic pH of that tissue (e.g., 7.1-7.4). Such tumor targeted agents may also be deactivated by the pH of CSF surrounding the tumor (e.g., 7.28 to 7.32). For ease of discussion such CSF will be defined to be part of the healthy tissue surrounding the tumor. The cytotoxic effect which is deactivated by exposure to pH of normal healthy tissue may include effects which interfere with or hinder cell replication including DNA replication. In such embodiments, the cytotoxic effect may include inhibition or interference with topoisomerase enzymes, herein topoisomerases including topoisomerase I. Agents which inhibit topoisomerases including topoisomerase I are known as topoisomerase inhibitors and topoisomerase I inhibitors respectively (the latter being a subset of the former). Examples of topoisomerase I inhibitors include lamellarin D, camptothecin and its analogues such as topotecan, irinotecan (and its active metabolite, 10, 11-methylenedioxy-CPT (MDC), and the alkylating derivative, 7-chloromethyl-10, 11-MDC. While the latter two of these compounds are somewhat more stable, all of these analogues are nonetheless degraded in the pH of health tissue including that of health brain tissue. This is due to that fact that all have an unstable lactone on the e-ring of the molecule that reversibly forms a hydroxy acid at physiological pH. The lactone being the active form and the hydroxyl acid being inactive. Since the reaction is reversible, when the molecule finds itself in an acidic environment such as that in a tumor in particular in a brain tumor it will either stay in active form or revert to active form.

One prime example of a tumor topoisomerase I inhibitor which is degraded in the pH of healthy tissue as described above is topotecan, and another is irinotecan. In order to maintain its tumor targeted capability, embodiments of medicinal solutions containing topotecan contemplated by the present disclosure contain little or substantially no buffering agent (e.g., less than 5% by molarity or volume, more preferably less than 1% still more preferably less than 0.25%). In particular embodiments of medicinal solutions including topotecan or other camptothecin analogue, the solution contains no, little, or substantially no acidic buffering agents including one or more of tartaric acid, citric acid, malic acid or monosodium citrate. Such targeted solutions are highly novel in that they are considered contrary to the teaching in the pharmaceutical arts where nearly all parenteral pharmaceutical solutions (e.g., IV or subcutaneous) contain buffering agents for the purpose of stabilizing the solution as well as maintaining the pH of the solution within a normal physiologic range. In particular with regard to topotecan, the art (specifically the manufactures packaging insert per the FDA) teaches that topotecan solutions contain tartaric acid, a known acidic buffering agent.

The advantage of such tumor targeted agents and solutions is that they have a cytotoxic effect on cancerous issue but have little or no toxic effect on healthy tissue because they become deactivated by the pH found within healthy tissue. This in turn allows for the delivery of higher concentrations and associated doses of the targeted agent to the tumor volume to produce a greater and faster cytotoxic effect with little or no toxic effect on surrounding healthy brain tissue.

In an embodiment of a method for using the tumor targeted solution containing tumor targeted agents, a medicinal solution including at least one active agent is intracranially administered into a TTS in a brain tumor of the subject using an embodiment of the intracranial drug delivery system described above. It then diffuses into the tumor volume where it has a cytotoxic effect on the cancer cells in the tumor. As the solution starts to diffuse into healthy tissue it becomes chemically deactivated by the pH in healthy tissue, losing its cytotoxic or other toxic effect on healthy tissue. Such deactivation allows the targeted solution to be infused (either continuously or intermittently) into the tumor for extended periods of time to produce enhanced cytotoxic effects against the tumor cells with minimal or no adverse effect on healthy tissue. In an embodiment, the catheter may contain pH sensors at or near its distal tip to allow for determination of the pH in the TTS. Information from the pH sensors can then be used to control or titrate one or more of the infusion flow rate, regimen or total amount of medicinal solution delivered so as to maintain the optimum pH for keeping the tumor targeted agent in its active (e.g., cytotoxic) form. In use, these and other embodiments of such methods provide the advantage of producing a prolonged and/or enhanced cytotoxic effect on the tumor and thus faster tumor shrinkage while minimizing adverse effects on healthy brain tissue.

The foregoing description of various embodiments has been presented for purposes of illustration and description. It is not intended to limit the invention to the precise forms disclosed. Many modifications, variations and refinements will be apparent to practitioners skilled in the art. For example, embodiments of the device can be sized and otherwise adapted for various pediatric and neonatal applications as well as various veterinary applications. Also, those skilled in the art will recognize, or be able to ascertain using no more than routine experimentation, numerous equivalents to the specific devices and methods described herein. Such equivalents are considered to be within the scope of the present invention and are covered by the appended claims below.

While the present disclosure has been described and illustrated with reference to specific embodiments thereof, these descriptions and illustrations do not limit the present disclosure. It can be clearly understood that various changes can be made, and equivalent components can be substituted within the embodiments, without departing from the true spirit and scope of the present disclosure as defined by the appended claims. Also, components, characteristics, or acts from one embodiment can be readily recombined or substituted with one or more components, characteristics or acts from other embodiments to form numerous additional embodiments within the scope of the invention. Moreover, components that are shown or described as being combined with other components, can, in various embodiments, exist as standalone components. Further, for any positive recitation of a component, characteristic, constituent, feature, step or the like, embodiments of the invention specifically contemplate the exclusion of that component, value, characteristic, constituent, feature, step or the like. The illustrations may not necessarily be drawn to scale. There can be distinctions between the artistic renditions in the present disclosure and the actual apparatus, due to variables in manufacturing processes and such. There can be other embodiments of the present disclosure which are not specifically illustrated. The specification and drawings are to be regarded as illustrative rather than restrictive. Modifications can be made to adapt a particular situation, material, composition of matter, method, or process to the objective, spirit and scope of the present disclosure. All such modifications are intended to be within the scope of the claims appended hereto. While the methods disclosed herein have been described with reference to particular operations performed in a particular order, it can be understood that these operations can be combined, sub-divided, or re-ordered to form an equivalent method without departing from the teachings of the present disclosure. Therefore, unless specifically indicated herein, the order and grouping of the operations are not limitations of the present disclosure.

1. A system for delivery of a medicinal solution to a tissue site in a brain of a subject, the system comprising:
a catheter having a proximal end and a distal end, the catheter defining at least one catheter lumen;
a cranial burr hole stopple, the stopple defining a stopple opening structured for advancement of the catheter therethrough, the stopple comprising:
a plug structured to be inserted into a burr hole in a cranium of the subject, the plug comprising at least one seal positioned on a wall of the stopple opening to form a fluidic seal with an exterior of the catheter; and
a flange structured to engage an outer surface of a skull of the subject when the plug is inserted into the burr hole, the flange defining a flange opening on a side portion of the flange and defining at least one groove on a top portion of the flange, the at least one groove structured to engage and retain the catheter;[[ and]]
a connecting member having a proximal end and a distal end, the distal end structured to be coupled to the proximal end of the catheter, the connecting member defining at least one connecting member lumen;
an anchoring element engaging the flange opening and structured to secure the connecting member to the flange; and
a connector tube having a proximal end and a distal end, the distal end structured to be coupled to the connecting member proximal end, the connector tube defining at least one connector tube lumen;
wherein the at least one catheter lumen, the at least one connecting member lumen, and the at least one connector tube lumen are structured to provide at least one flow path, when assembled together, for delivery of the medicinal solution to the tissue site in the brain.

2. The system of claim 1, further comprising
a pump having an outlet structured to be coupled to the proximal end of the connector tube, the pump structured to pump the medicinal solution through the flow path; and
a reservoir for storage of the medicinal solution, the reservoir fluidically coupled to the pump.

3. The system of claim 1, wherein the at least one catheter lumen is sized and structured for advancement of a positioning stylette therethrough.

4. (canceled)

5. The system of claim 1, wherein the catheter has a durometer in a range of 20-30.

6. The system of claim 1, further comprising a one-way valve to prevent backflow of fluids from the brain into the flow path.

7. The system of claim 1, wherein a distal portion of the catheter includes a plurality of apertures fluidically coupled to the at least one catheter lumen for delivery of the medicinal fluid to the tissue site, the plurality of apertures arranged in a pattern and structured to produce a target diffusion volume.

8. The system of claim 1, wherein the catheter comprises a distal portion having a first durometer and a remainder portion having a second durometer greater than the first durometer.

9. The system of claim 1, further comprising a pressure sensor structured to provide information on a. flow rate of medicinal solution through the flow path.

10. The system of claim 1, further comprising a pressure sensor structured and positioned on or in the catheter to detect pressure within brain tissue near or adjacent to a distal tip of the catheter,

11. The system of claim 1, further comprising circuitry disposed on or within the burr hole stopple.

12. A method for the intracranial delivery of an active agent to a brain to focally treat a growth in a cranium of a subject, the method comprising
advancing a catheter through a sealable opening in a burr hole stopple positioned in a burr hole in the cranium the burr hole stopple such that a tip of the catheter is positioned at a tissue site in the brain within or in proximity to the growth, the catheter defining a fluidic lumen, the catheter further defining at least one aperture fluidically coupled to the fluidic lumen at a distal portion of the catheter;
affixing a proximal portion of the catheter to a fixation feature in the burr hole stopple;
creating a flow path between the tissue site and a pump operatively coupled to a reservoir containing a medicinal solution comprising the active agent; and
pumping the medicinal solution from the reservoir to the tissue site via the flow path; wherein a flow rate and a duration of delivery are selected based on an established model so as to achieve a selected steady state diffusion volume of medicinal solution in the brain tissue.

13. The method of claim 12, where advancing the catheter comprises:
advancing a stylette through the burr hole stopple opening to the tissue site in the brain;
advancing the catheter over the stylette such that a tip of the catheter is positioned at or in the tissue site; and
removing the stylette.

14. The method of claim 12, wherein the medicinal solution is delivered to the tissue site in a regimen comprising at least one on period and at least one off period.

15. The method of claim 12, wherein the model is established based on brain imaging observations of diffusion volume using infusion of contrast agents at various flow rates over time.

16. The method of claim 12, wherein the selected steady state diffusion volume is larger than a volume of the growth to treat a selected healthy tissue margin surrounding the growth. 17, (original) The method of claim 16, wherein the active agent comprises a chemotherapeutic agent having an active form and an inactive form and wherein the chemotherapeutic agent remains in the active form in cancerous brain tissue and converts to the inactive form in healthy brain tissue.

18. The method of claim 17, wherein the chemotherapeutic agent comprises topotecan.

19. A method fur focally treating a brain growth, the method comprising:
intracranially delivering to a tissue site in the brain, within or in proximity to the growth, a medicinal solution comprising an active agent which is degraded at a pH at or above that found in healthy brain tissue, wherein the solution comprises substantially no buffering agent; and
wherein the active agent has a cytotoxic effect on cancerous tissue in the growth and is deactivated upon contact with or after entering into healthy brain tissue surrounding the growth.

20. The method of claim 19, wherein the solution has a pH greater than about 2.5 and less than about 4.

21. The method of claim 19, wherein the active agent comprises topotecan or its analogues and derivatives.
